(19) United States
(12) Patent Application Publication
(10) Pub. No.: US 2011/0201615 A1
(43) Pub. Date: Aug. 18, 2011

(54) TRIAZOLE BETA CARBOLINE DERIVATIVES AS ANTI-DIABETIC AGENTS

(76) Inventors: Liangqin Guo, Edison, NJ (US); Jian Liu, Edison, NJ (US); Ravi P. Nargund, East Brunswick, NJ (US); Alexander Pasternak, Princeton, NJ (US); Lihu Yang, Edison, NJ (US); Zhixiong Ye, West Windsor, NJ (US)

(21) Appl. No.: 13/125,319

(22) PCT Filed: Oct. 19, 2009
(86) PCT No.: PCT/US09/61154

§ 371 Date: Apr. 21, 2011

Related U.S. Application Data
(60) Provisional application No. 61/197,597, filed on Oct. 29, 2008.

(51) Int. Cl. A61K 31/501; C07D 471/04; A61K 31/437; C07D 401/14; A61K 31/506; A61P 3/00; A61P 9/12

(52) U.S. Cl. 514/252.04; 546/85; 514/292; 544/238; 544/333; 514/256

(57) ABSTRACT

Beta-carboline derivatives of structural formula (I) are selective antagonists of the somatostatin subtype receptor 3 (SSTR3) and are useful for the treatment of Type 2 diabetes mellitus and of conditions that are often associated with this disease, including hyperglycemia, insulin resistance, obesity, lipid disorders, and hypertension. The compounds are also useful for the treatment of depression and anxiety.

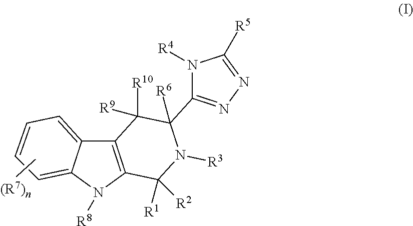

### FIELD OF THE INVENTION

The instant invention is concerned with substituted beta-carboline derivatives, which are selective antagonists of the somatostatin subtype receptor 3 (SSTR3) which are useful for the treatment of Type 2 diabetes mellitus and of conditions that are often associated with this disease, including hyperglycemia, insulin resistance, obesity, lipid disorders, and hypertension. The compounds are also useful for the treatment of depression and anxiety.

### BACKGROUND OF THE INVENTION

Diabetes is a disease derived from multiple causative factors and characterized by elevated levels of plasma glucose (hyperglycemia) in the fasting state or after administration of glucose during an oral glucose tolerance test. There are two generally recognized forms of diabetes. In type 1 diabetes, or insulin-dependent diabetes mellitus (IDDM), patients produce little or no insulin, the hormone which regulates glucose utilization. In Type 2 diabetes, or noninsulin-dependent diabetes mellitus (NIDDM), insulin is still produced by islet cells in the pancreas. Patients having Type 2 diabetes have a resistance to the effects of insulin in stimulating glucose and lipid metabolism in the main insulin-sensitive tissues, including muscle, liver and adipose tissues. These patients often have normal levels of insulin, and may have hyperinsulinemia (elevated plasma insulin levels), as they compensate for the reduced effectiveness of insulin by secreting increased amounts of insulin (Polonsky, Int. J. Obes. Relat. Metab. Disord 24 Suppl 2:S29-31, 2000). The beta cells within the pancreatic islets initially compensate for insulin resistance by increasing insulin output. Insulin resistance is not primarily caused by a diminished number of insulin receptors but rather by a post-insulin receptor binding defect that is not yet completely understood. This lack of responsiveness to insulin results in insufficient insulin-mediated activation of uptake, oxidation and storage of glucose in muscle, and inadequate insulin-mediated repression of lipolysis in adipose tissue and of glucose production and secretion in the liver. Eventually, a patient may be become diabetic due to the inability to properly compensate for insulin resistance. In humans, the onset of Type 2 diabetes due to insufficient increases (or actual declines) in beta cell mass is apparently due to increased beta cell apoptosis relative to non-diabetic insulin resistant individuals (Butler et al., Diabetes 52:102-110, 2003).

Persistent or uncontrolled hyperglycemia that occurs with diabetes is associated with increased and premature morbidity and mortality. Often abnormal glucose homeostasis is associated both directly and indirectly with obesity, hypertension, and alterations of the lipid, lipoprotein and apolipoprotein metabolism, as well as other metabolic and hemodynamic disease. Patients with Type 2 diabetes mellitus have a significantly increased risk of macrovascular and microvascular complications, including atherosclerosis, coronary heart disease, stroke, peripheral vascular disease, hypertension, nephropathy, neuropathy, and retinopathy. Therefore, effective therapeutic control of glucose homeostasis, lipid metabolism, obesity, and hypertension are critically important in the clinical management and treatment of diabetes mellitus. Patients who have insulin resistance often exhibit several symptoms that together are referred to as syndrome X or Metabolic Syndrome. According to one widely used definition, a patient having Metabolic Syndrome is characterized as having three or more symptoms selected from the following group of five symptoms: (1) abdominal obesity, (2) hypertriglyceridemia, (3) low levels of high-density lipoprotein cholesterol (HDL), (4) high blood pressure, and (5) elevated fasting glucose, which may be in the range characteristic of Type 2 diabetes if the patient is also diabetic. Each of these symptoms is defined clinically in the Third Report of the National Cholesterol Education Program Expert Panel on Detection, Evaluation and Treatment of High Blood Cholesterol in Adults (Adult Treatment Panel III, or ATP III), National Institutes of Health, 2001, NIH Publication No. 01-3670. Patients with Metabolic Syndrome, whether they have or develop overt diabetes mellitus, have an increased risk of developing the macrovascular and microvascular complications that occur with Type 2 diabetes, such as atherosclerosis and coronary heart disease.

There are several available treatments for Type 2 diabetes, each of which has its own limitations and potential risks. Physical exercise and a reduction in dietary intake of calories often dramatically improves the diabetic condition and are the usual recommended first-line treatment of Type 2 diabetes and of pre-diabetic conditions associated with insulin resistance. Compliance with this treatment is generally very poor because of well-entrenched sedentary lifestyles and excess food consumption, especially of foods containing high amounts of fat and carbohydrates. Pharmacologic treatments have largely focused on three areas of pathophysiology: (1) hepatic glucose production (biguanides), (2) insulin resistance (PPAR agonists), (3) insulin secretion (sulfonylureas); (4) incretin hormone mimetics (GLP-1 derivatives and analogs, such as exenatide and luraglitide); and (5) inhibitors of incretin hormone degradation (DPP-4 inhibitors).

The biguanides belong to a class of drugs that are widely used to treat Type 2 diabetes. Phenformin and metformin are the two best known biguanides and do cause some correction of hyperglycemia. The biguanides act primarily by inhibiting hepatic glucose production, and they also are believed to modestly improve insulin sensitivity. The biguanides can be used as monotherapy or in combination with other anti-diabetic drugs, such as insulin or insulin secretagogues, without increasing the risk of hypoglycemia. However, phenformin and metformin can induce lactic acidosis, nausea/vomiting, and diarrhea. Metformin has a lower risk of side effects than phenformin and is widely prescribed for the treatment of Type 2 diabetes.

The glitazones (e.g., 5-benzylthiazolidine-2,4-diones) are a class of compounds that can ameliorate hyperglycemia and other symptoms of Type 2 diabetes. The glitazones that are currently marketed (rosiglitazone and pioglitazone) are agonists of the peroxisome proliferator activated receptor (PPAR) gamma subtype. The PPAR-gamma agonists substantially increase insulin sensitivity in muscle, liver and adipose tissue in several animal models of Type 2 diabetes, resulting in partial or complete correction of elevated plasma glucose levels without the occurrence of hypoglycemia. PPAR-gamma agonism is believed to be responsible for the improved insulin sensititization that is observed in human patients who are treated with the glitazones. New PPAR agonists are currently being developed. Many of the newer PPAR compounds are agonists of one or more of the PPAR alpha, gamma and delta subtypes. The currently marketed PPAR gamma agonists are modestly effective in reducing plasma glucose and hemoglobinA1C. The currently marketed compounds do not greatly improve lipid metabolism and may actually have a negative effect on the lipid profile. Thus, the PPAR compounds represent an important advance in diabetic therapy.

Another widely used drug treatment involves the administration of insulin secretagogues, such as the sulfonylureas (e.g., tolbutamide, glipizide, and glimepiride). These drugs increase the plasma level of insulin by stimulating the pancreatic O-cells to secrete more insulin. Insulin secretion in the pancreatic β-cell is under strict regulation by glucose and an array of metabolic, neural and hormonal signals. Glucose stimulates insulin production and secretion through its metabolism to generate ATP and other signaling molecules, whereas other extracellular signals act as potentiators or inhibitors of insulin secretion through GPCR's present on the plasma membrane. Sulfonylureas and related insulin secretagogues act by blocking the ATP-dependent K+ channel in O-cells, which causes depolarization of the cell and the opening of the voltage-dependent Ca2+ channels with stimulation of insulin release. This mechanism is non-glucose dependent, and hence insulin secretion can occur regardless of the ambient glucose levels. This can cause insulin secretion even if the glucose level is low, resulting in hypoglycemia, which can be fatal in severe cases. The administration of insulin secretagogues must therefore be carefully controlled. The insulin secretagogues are often used as a first-line drug treatment for Type 2 diabetes.

Dipeptidyl peptidase-IV (DPP-4) inhibitors (e.g., sitagliptin, vildagliptin, saxagliptin, and alogliptin) provide a new route to increase insulin secretion in response to food consumption. Glucagon-like peptide-1 (GLP-1) levels increase in response to the increases in glucose present after eating and glucagon stimulates the production of insulin. The serine proteinase enzyme DPP-4 which is present on many cell surfaces degrades GLP-1. DPP-4 inhibitors reduce degradation of GLP-1, thus potentiating its action and allowing for greater insulin production in response to increases in glucose through eating.

There has been a renewed focus on pancreatic islet-based insulin secretion that is controlled by glucose-dependent insulin secretion. This approach has the potential for stabilization and restoration of β-cell function. In this regard, the present application claims compounds that are antagonists of the somatostatin subtype receptor 3 (SSTR3) as a means to increase insulin secretion in response to rises in glucose resulting from eating a meal. These compounds may also be used as ligands for imaging (e.g., PET, SPECT) for assessment of beta cell mass and islet function. A decrease in β-cell mass can be determined with respect to a particular patient over the course of time.

### SUMMARY OF THE INVENTION

The present invention is directed to compounds of structural formula I, and pharmaceutically acceptable salts thereof:

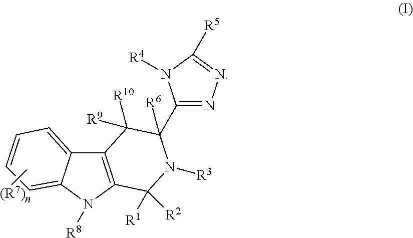

These bicyclic beta-carboline derivatives are effective as antagonists of SSTR3. They are therefore useful for the treatment, control and prevention of disorders responsive to antagonism of SSTR3, such as Type 2 diabetes, insulin resistance, lipid disorders, obesity, atherosclerosis, Metabolic Syndrome, depression, and anxiety.

The present invention also relates to compositions comprising the compounds of the present invention and a pharmaceutically acceptable carrier.

The present invention also relates to methods for the treatment, control, or prevention of disorders, diseases, or conditions responsive to antagonism of SSTR3 in a subject in need thereof by administering the compounds and compositions of the present invention.

The present invention also relates to methods for the treatment, control, or prevention of Type 2 diabetes, hyperglycemia, insulin resistance, obesity, lipid disorders, atherosclerosis, and Metabolic Syndrome by administering the compounds and compositions of the present invention.

The present invention also relates to methods for the treatment, control, or prevention of depression and anxiety by administering the compounds and pharmaceutical compositions of the present invention.

The present invention also relates to methods for the treatment, control, or prevention of obesity by administering the compounds of the present invention in combination with a therapeutically effective amount of another agent known to be useful to treat the condition.

The present invention also relates to methods for the treatment, control, or prevention of Type 2 diabetes by administering the compounds of the present invention in combination with a therapeutically effective amount of another agent known to be useful to treat the condition.

The present invention also relates to methods for the treatment, control, or prevention of atherosclerosis by administering the compounds of the present invention in combination with a therapeutically effective amount of another agent known to be useful to treat the condition.

The present invention also relates to methods for the treatment, control, or prevention of lipid disorders by administering the compounds of the present invention in combination with a therapeutically effective amount of another agent known to be useful to treat the condition.

The present invention also relates to methods for treating Metabolic Syndrome by administering the compounds of the present invention in combination with a therapeutically effective amount of another agent known to be useful to treat the condition.

The present invention also relates to methods for the treatment, control, or prevention of depression and anxiety by administering the compounds of the present invention in combination with a therapeutically effective amount of another agent known to be useful to treat the condition.

### DETAILED DESCRIPTION OF THE INVENTION

The present invention is concerned with beta-carboline derivatives useful as antagonists of SSTR3. Compounds of the present invention are described by structural formula I:

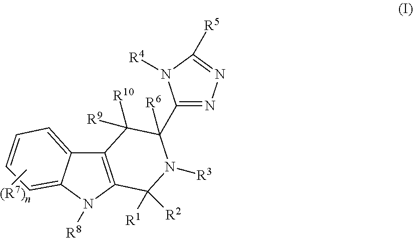

or a pharmaceutically acceptable salt thereof, wherein:
R1 is selected from the group consisting of:
(1) C₁₋₁₀ alkyl,
(2) —(O)OR^{e},
(3) —C(O)NR^{c}R^{d},
(4) C₂₋₁₀ cycloheteroalkyl,
(5) C₂₋₁₀ cycloheteroalkyl-C₁₋₁₀ alkyl-,
(6) aryl,
(7) heteroaryl, and
(8) heteroaryl-C₁₋₁₀ alkyl-;
   wherein alkyl and cycloheteroalkyl are optionally substituted with one to three substituents independently selected from R^{a}, and aryl and heteroaryl are optionally substituted with one to three substituents independently selected from R^{b};
   R2 is selected from the group consisting of

(1) hydrogen,

(2) C₁₋₁₀ alkyl,

(3) C₂₋₁₀ alkenyl,

(4) C₂₋₁₀ alkynyl,

(5) C₃₋₁₀ cycloalkyl,

(6) C₃₋₁₀ cycloalkyl-C₁₋₁₀ alkyl-,

(7) C₁₋₆ alkyl-X—C₁₋₆ alkyl-,

(8) C₃₋₁₀ cycloalkyl-X—C₁₋₆ alkyl-,

(9) C₂₋₁₀ cycloheteroalkyl,

(10) aryl,

(11) heteroaryl,

(12) heteroaryl-C₁₋₆ alkyl-,

(13) aryl-C₁₋₄ alkyl-X—C₁₋₄ alkyl-, and

(14) heteroaryl-C₁₋₄ alkyl-X—C₁₋₄ alkyl-,

wherein X is selected from the group consisting of oxygen, sulfur, and NR⁴, and alkyl, alkenyl, alkynyl are optionally substituted with one to three substituents independently selected from R^{a}, and cycloalkyl, cycloheteroalkyl, aryl and heteroaryl are optionally substituted with one to three substituents independently selected from R^{b};
R³ is selected from the group consisting of

(1) hydrogen,

(2) —C_{1-—}10 alkyl,

(3) —C₃₋₁₀ cycloalkyl,

(4) C₂₋₁₀ cycloheteroalkyl,

(5) C₂₋₁₀ cycloheteroalkyl-C₁₋₆ alkyl-, and

(6) heteroaryl-C₁₋₆ alkyl-,

wherein alkyl, cycloalkyl, and cycloheteroalkyl are optionally substituted with one to three substituents independently selected from R^{a}, and heteroaryl is optionally substituted with one to three substituents independently selected from R^{b};
R⁴ is selected from:

(1) hydrogen, and

(2) —C₁₋₁₀ alkyl, optionally substituted with one to five fluorines;

R⁵ is independently selected from the group consisting of

(1) hydrogen,

(2) —C₁₋₁₀ alkyl,

(3) —C₂₋₁₀ alkenyl,

(4) —C₂₋₁₀ alkynyl,

(5) —C₃₋₁₀ cycloalkyl,

(6) C₂₋₁₀ cycloheteroalkyl,

(7) aryl, and

(8) heteroaryl,

wherein alkyl, alkenyl, alkynyl, cycloalkyl, and cycloheteroalkyl are optionally substituted with one to three substituents independently selected from R^{a}, and aryl and heteroaryl are optionally substituted with one to three substituents independently selected from R^{b};
R⁶ is selected from the group consisting of:

(1) hydrogen,

(2) —C₁₋₁₀ alkyl, optionally substituted with one to five fluorines,

(3) —C₂₋₁₀ alkenyl,

(4) —C₃₋₁₀ cycloalkyl, and

(5) —C₁₋₄ alkyl-O—C₁₋₄ alkyl-;

each R⁷ is independently selected from the group consisting of:
(1) hydrogen,
(2) —OR^{e},
(3) —NR^{c}S(O)ₘR^{e},
(4) halogen,
(5) —S(O)ₘR^{e},
(6) —S(O)ₘNR^{c}R^{d},
(7) —NR^{c}R^{d},
(8) —C(O)R^{e},
(9) —OC(O)R^{e},
(10) —CO₂R^{e},
(11) —CN,
(12) —C(O)NR^{c}R^{d},
(13) —NR^{c}C(O)R^{e},
(14) —NR^{c}C(O)OR^{e},
(15) —NR^{c}C(O)NRCR^{d},
(16) —OCF₃,
(17) —OCH₂,
(18) C₂₋₁₀ cycloheteroalkyl,
(19) —C₁₋₁₀ alkyl, optionally substituted with one to five fluorines,
(20) —C₃₋₆ cycloalkyl,
(21) aryl, and
(22) heteroaryl,
   wherein aryl and heteroaryl are optionally substituted with one to three substituents independently selected from R^{b};
   R8 is selected from the group consisting of
(1) hydrogen,
(2) —C₁₋₁₀ alkyl,
(3) —C₂₋₁₀ alkenyl, and
(4) —C₃₋₁₀ cycloalkyl,
   wherein alkyl, alkenyl, and cycloalkyl are optionally substituted with one to three substituents independently selected from R^{a};
   R⁹ and R¹⁰ are each independently selected from:
(1) hydrogen, and
(2) —C₁₋₄ alkyl, optionally substituted with one to five fluorines;
   each R^{a} is independently selected from the group consisting of:
(1) —OR^{e},
(2) —NR^{c}S(O)ₘR^{e},
(3) halogen,
(4) —S(O)ₘR^{e},
(5) —S(O)ₘNR^{c}R^{d},
(6) —NR^{c}R^{d},
(7) —C(O)R^{e},
(8) —OC(O)R^{e},
(9) oxo,
(10) —CO₂R^{e},
(11) —CN,
(12) —C(O)NR^{c}R^{d},
(13) —NR^{c}C(O)R^{e},
(14) —NR^{c}C(O)OR^{e},
(15) —NR^{c}C(O)NR^{c}R^{d},
(16) —CF₃,
(17) —OCF₃,
(18) —OCHF₂, and
(19) C₂₋₁₀ cycloheteroalkyl;
   each R^{b} is independently selected from the group consisting of:
(1) R^{a},
(2) C₁₋₁₀ alkyl, and
(3) C₃₋₆ cycloalkyl;
   R^{c} and R^{d} are each independently selected from the group consisting of:

(1) hydrogen,

(2) —C₁₋₁₀ alkyl,

(3) —C₂₋₁₀ alkenyl,

(4) —C₃₋₆ cycloalkyl,

(5) —C₃₋₆ cycloalkyl-C₁₋₁₀ alkyl-,

(6) C₂₋₁₀ cycloheteroalkyl,

(7) C₂₋₁₀ cycloheteroalkyl-C₁₋₁₀ alkyl-,

(8) aryl,

(9) heteroaryl,

(10) aryl-C₁₋₁₀ alkyl-, and

(11) heteroaryl-C₁₋₁₀ alkyl-, or

R^{c} and R^{d} together with the atom(s) to which they are attached form a heterocyclic ring of 4 to 7 members containing 0-2 additional heteroatoms independently selected from oxygen, sulfur and N—R^{g} when R^{c} and R^{d} are other than hydrogen, and wherein each R^{c} and R^{d} is optionally substituted with one to three substituents independently selected from R^{h};
each R^{e} is independently selected from the group consisting of:
(1) hydrogen,
(2) —C₁₋₁₀ alkyl,
(3) —C₂₋₁₀ alkenyl,
(4) —C₃₋₆ cycloalkyl,
(5) —C₃₋₆ cycloalkyl-C₁₋₁₀ alkyl-,
(6) C₂₋₁₀ cycloheteroalkyl,
(7) C₂₋₁₀ cycloheteroalkyl-C₁₋₁₀ alkyl-,
(8) aryl,
(9) heteroaryl,
(10) aryl-C_{I}-10 alkyl-, and
(11) heteroaryl-C₁₋₁₀ alkyl-,
   wherein when R^{e} is not hydrogen, each R^{e} is optionally substituted with one to three substituents selected from R^{h};
   each R^{g} is independently selected from:
(1) —C(O)R^{e}, and
(2) —C₁₋₁₀ alkyl, optionally substituted with one to five fluorines;
   each R^{h} is independently selected from the group consisting of:
(1) halogen,
(2) —C₁₋₁₀ alkyl,
(3) —O—C₁₋₄ alkyl,
(4) —S(O)ₘ—C₁₋₄ alkyl,
(5) —CN,
(6) —CF₃,
(7) —OCHF₂, and
(8) —OCF₃;
   each m is independently 0, 1 or 2; and
   each n is independently 0, 1, 2 or 3.

The invention has numerous embodiments, which are summarized below. The invention includes compounds of Formula I. The invention also includes pharmaceutically acceptable salts of the compounds and pharmaceutical compositions comprising the compounds and a pharmaceutically acceptable carrier. The compounds are useful for the treatment of Type 2 diabetes, hyperglycemia, obesity, and lipid disorders that are associated with Type 2 diabetes.

In one embodiment of the compounds of the present invention, R¹ is selected from the group consisting of: —C₁₋₁₀ alkyl, —C(O)OR^{e}, —C(O)NR^{c}R^{d}, C₂₋₁₀ cycloheteroalkyl, C₂₋₁₀ cycloheteroalkyl-C₁₋₁₀ alkyl-, aryl, heteroaryl, and heteroaryl-C₁₋₁₀ wherein alkyl and cycloheteroalkyl are unsubstituted or substituted with one to three substituents independently selected from R^{a}; and aryl and heteroaryl are unsubstituted or substituted with one to three substituents independently selected from R^{b}. In a class of this embodiment, R¹ is selected from the group consisting of: C₁₋₁₀ alkyl, aryl, and heteroaryl, wherein alkyl is unsubstituted or substituted with one to three substituents independently selected from R^{a}; and aryl and heteroaryl are unsubstituted or substituted with one to three substituents independently selected from R^{b}. In a subclass of this class, R¹ is selected from the group consisting of —(CH₂)₃CH₃, phenyl, oxadiazole, pyrazole, pyridine, furan, pyrimidine, and pyridazine, wherein alkyl is unsubstituted or substituted with one to three substituents independently selected from R^{a}; and aryl and heteroaryl are unsubstituted or substituted with one to three substituents independently selected from R^{b}. In another subclass of this class, R¹ is selected from the group consisting of: —(CH₂)₃CH₃, phenyl, oxadiazole, pyrazole, pyridine, furan, pyrimidine, and pyridazine, wherein alkyl is unsubstituted or substituted with one to three substituents independently selected from: halogen and CN; and aryl and heteroaryl are unsubstituted or substituted with one to three substituents independently selected from: —C₁₋₆ alkyl, and halogen. In another subclass of this class, R¹ is selected from the group consisting of: oxadiazole, pyrazole, furan and pyridine, wherein heteroaryl is unsubstituted or substituted with one to three substituents independently selected from: —C₁₋₆ alkyl, and halogen. In another class of this embodiment, R¹ is heteroaryl, wherein heteroaryl is unsubstituted or substituted with one to three substituents independently selected from R^{b}.

In another embodiment of the present invention, R² is selected from the group consisting of: hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₁₀ alkyl-, C₁₋₆ alkyl-X—C₁₋₆ alkyl-, C₃₋₁₀ cycloalkyl-X—C₁₋₆ alkyl-, C₂₋₁₀ cycloheteroalkyl, aryl, heteroaryl, heteroaryl-C₁₋₆ alkyl, aryl-C₁₋₄ alkyl-X—C₁₋₄ alkyl-, and heteroaryl-C₁₋₄ alkyl-X—C₁₋₄ alkyl-, wherein X is selected from the group consisting of oxygen, sulfur, and NR⁴, and wherein alkyl, alkenyl, alkynyl are unsubstituted or substituted with one to three substituents independently selected from R^{a}; and cycloalkyl, cycloheteroalkyl, aryl and heteroaryl are unsubstituted or substituted with one to three substituents independently selected from R^{b}. In a class of this embodiment, R² is selected from the group consisting of hydrogen, C₁₋₁₀ alkyl, C₃₋₁₀ cycloalkyl, C₂₋₁₀ cycloheteroalkyl, aryl, and heteroaryl, wherein alkyl is unsubstituted or substituted with one to three substituents independently selected from R^{a}; and cycloalkyl, cycloheteroalkyl, aryl and heteroaryl are unsubstituted or substituted with one to three substituents independently selected from R^{b}. In a subclass of this class, R² is selected from the group consisting of hydrogen, —(CH₂)₃CH₃, —CH₂CN, cyclohexane, tetrahydropyran, phenyl, pyrazole, furan, pyrimidine, pyridazine, pyridine, and oxadiazole, wherein alkyl is unsubstituted or substituted with one to three substituents independently selected from R^{a}; and cycloalkyl, cycloheteroalkyl, aryl and heteroaryl are unsubstituted or substituted with one to three substituents independently selected from R^{b}. In another class of this embodiment, R² is selected from the group consisting of hydrogen, C₁₋₁₀ alkyl, aryl, and heteroaryl, wherein alkyl is unsubstituted or substituted with one to three substituents independently selected from R^{a}; and aryl and heteroaryl are unsubstituted or substituted with one to three substituents independently selected from R^{b}. In a subclass of this class, R² is selected from the group consisting of: hydrogen, —(CH₂)₃CH₃, —CH₂CN, phenyl, pyrazole, furan, pyrimidine, pyridazine, pyridine, and oxadiazole, wherein alkyl is unsubstituted or substituted with one to three substituents independently selected from R^{a}; and phenyl and heteroaryl are unsubstituted or substituted with one to three substituents independently selected from R^{b}. In another subclass of this class, R² is selected from the group consisting of —(CH₂)₃CH₃, phenyl, and pyrazole, wherein alkyl is unsubstituted or substituted with one to three substituents independently selected from R^{a}; and phenyl and pyrazole are unsubstituted or substituted with one to three substituents independently selected from R^{b}. In another class of this embodiment, R² is selected from the group consisting of C₁₋₁₀ alkyl, aryl, and heteroaryl, wherein alkyl is unsubstituted or substituted with one to three substituents independently selected from R^{a}; and aryl and heteroaryl are unsubstituted or substituted with one to three substituents independently selected from R^{b}. In a subclass of this class, R² is selected from the group consisting of: C₁₋₁₀ alkyl, phenyl and heteroaryl, wherein alkyl is unsubstituted or substituted with one to three substituents independently selected from R^{a}; and phenyl and heteroaryl are unsubstituted or substituted with one to three substituents independently selected from R^{b}. In another subclass of this class, R² is selected from the group consisting of: —(CH₂)₃CH₃, —CH₂CN, phenyl, pyrazole, furan, pyrimidine, pyridazine, pyridine, and oxadiazole, wherein alkyl is unsubstituted or substituted with one to three substituents independently selected from R^{a}, and aryl and heteroaryl are unsubstituted or substituted with one to three substituents independently selected from R^{b}. In another subclass of this class, R² is selected from the group consisting of: —(CH₂)₃CH₃, phenyl, and pyrazole, wherein alkyl is unsubstituted or substituted with one to three substituents independently selected from R^{a}; and phenyl and pyrazole are unsubstituted or substituted with one to three substituents independently selected from R^{b}. In another subclass of this class, R² is selected from the group consisting of: —(CH₂)₃CH₃, phenyl, and pyrazole, wherein alkyl is unsubstituted or substituted with one to three substituents independently selected from R^{a}, and phenyl and pyrazole are unsubstituted or substituted with one to three substituents independently selected from: C₁₋₁₀ alkyl and halogen.

In another embodiment of the present invention, R² is selected from the group consisting of: C₁₋₁₀ alkyl, C₂₋₆ cycloheteroalkyl, aryl, and heteroaryl, wherein alkyl is unsubstituted or substituted with one to three substituents independently selected from R^{a}, and cycloheteroalkyl, aryl and heteroaryl are unsubstituted or substituted with one to three substituents independently selected from R^{b}. In a subclass of this class, R² is selected from the group consisting of C₁₋₁₀ alkyl, C₂₋₆ cycloheteroalkyl, phenyl and heteroaryl, wherein alkyl is unsubstituted or substituted with one to three substituents independently selected from R^{a}; and phenyl and heteroaryl are unsubstituted or substituted with one to three substituents independently selected from R^{b}. In another subclass of this class, R² is selected from the group consisting of: —(CH₂)₃CH₃, —CH₂CN, phenyl, pyrazole, furan, tetrahydropyran, pyrimidine, pyridazine, pyridine, and oxadiazole, wherein alkyl is unsubstituted or substituted with one to three substituents independently selected from R^{a}, and cycloheteroalkyl, aryl and heteroaryl are unsubstituted or substituted with one to three substituents independently selected from R^{b}. In another subclass of this class, R² is selected from the group consisting of —(CH₂)₃CH₃, phenyl, pyridine, tetrahydropyran and pyrazole, wherein alkyl is unsubstituted or substituted with one to three substituents independently selected from R^{a}; and tetrahydropyran, phenyl and pyrazole are unsubstituted or substituted with one to three substituents independently selected from R^{b}. In another subclass of this class, R² is selected from the group consisting of: —(CH₂)₃CH₃, phenyl, tetrahydropyran, pyridine and pyrazole, wherein alkyl is unsubstituted or substituted with one to three substituents independently selected from R^{a}, and tetrahydropyran, phenyl, pyridine and pyrazole are unsubstituted or substituted with one to three substituents independently selected from: C₁₋₁₀ alkyl and halogen. In another embodiment of the present invention, R² is hydrogen.

In another embodiment of the present invention, R³ is selected from the group consisting of: hydrogen, —C₁₋₁₀ alkyl, —C₃₋₁₀ cycloalkyl, C₂₋₁₀ cycloheteroalkyl, C₂₋₁₀ cycloheteroalkyl-C₁₋₆ alkyl-, and heteroaryl-C₁₋₆ alkyl-, wherein alkyl, cycloalkyl, and cycloheteroalkyl are unsubstituted or substituted with one to three substituents independently selected from R^{a}; and heteroaryl is unsubstituted or substituted with one to three substituents independently selected from R^{b}. In a class of this embodiment, R³ is selected from the group consisting of: hydrogen, and —C₁₋₁₀ alkyl, wherein alkyl is unsubstituted or substituted with one to three substituents independently selected from R^{a}. In another class of this embodiment, R³ hydrogen.

In another embodiment of the present invention, R⁴ is selected from: hydrogen and —C₁₋₁₀ alkyl, wherein alkyl is unsubstituted or substituted with one to five fluorines. In a class of the embodiment, R⁴ is hydrogen. In another class of the embodiment, R⁴ is —C₁₋₁₀ alkyl, wherein alkyl is unsubstituted or substituted with one to five fluorines.

In another embodiment of the present invention, R⁵ is independently selected from the group consisting of hydrogen, —C₁₋₁₀ alkyl, —C₂₋₁₀ alkenyl, —C₂₋₁₀ alkynyl, —C₃₋₁₀ cycloalkyl, C₂₋₁₀ cycloheteroalkyl, aryl, and heteroaryl, wherein alkyl, alkenyl, alkynyl, cycloalkyl, and cycloheteroalkyl are unsubstituted or substituted with one to three substituents independently selected from R^{a}, and aryl and heteroaryl are unsubstituted or substituted with one to three substituents independently selected from R^{b}. In a class of this embodiment, R⁵ is independently selected from the group consisting of: aryl, and heteroaryl, wherein aryl and heteroaryl are unsubstituted or substituted with one to three substituents independently selected from R^{b}. In another class of this embodiment, R⁵ is aryl, wherein aryl is unsubstituted or substituted with one to three substituents independently selected from R^{b}. In a subclass of this class, R⁵ is phenyl, wherein phenyl is unsubstituted or substituted with one to three substituents independently selected from halogen. In another subclass of this class, R⁵ is phenyl, wherein phenyl is unsubstituted or substituted with one to three fluorines. In another subclass of this class, R⁵ is selected from the group consisting of: phenyl, para-fluorophenyl, and meta-fluorophenyl.

In another embodiment of the present invention, R⁶ is selected from the group consisting of: hydrogen, —C₁₋₁₀ alkyl, —C₂₋₁₀ alkenyl, —C₃₋₁₀ cycloalkyl, and —C₁₋₄ alkyl-O—C₁₋₄ alkyl-, wherein alkyl is unsubstituted or substituted with one to five fluorines. In a class of this embodiment, R⁶ is selected from the group consisting of: hydrogen, and —C₁₋₁₀ alkyl, wherein alkyl is unsubstituted or substituted with one to five fluorines. In another class of this embodiment, R⁶ is hydrogen.

In another embodiment of the present invention, each R⁷ is independently selected from the group consisting of hydrogen, —OR^{e}, —NR^{c}S(O) R^{e}, halogen, —S(O)ₘR^{e}, —S(O)ₘNR^{c}R^{d}, —NR^{c}R^{d}, —C(O)R^{e}, —OC(O)R^{e}, —CO₂R^{e}, —CN, —C(O)NR^{c}R^{d}, —NR^{c}C(O)R^{e}, —NR^{c}C(O)OR^{e}, —NR^{c}C(O)NR^{c}R^{d}, —OCF₃, —OCHF₂, C₂₋₆cycloheteroalkyl, —C₁₋₁₀ alkyl, optionally substituted with one to five fluorines, —C₃₋₆ cycloalkyl, aryl, and heteroaryl, wherein alkyl is unsubstituted or substituted with one to five fluorines, and wherein aryl and heteroaryl are unsubstituted or substituted with one to three substituents independently selected from R^{b}. In a class of this embodiment, each R⁷ is independently selected from the group consisting of: hydrogen, halogen, and —CN. In a subclass of this class, each R⁷ is independently selected from the group consisting of: hydrogen, Cl, F and CN. In another class of this embodiment, each R⁷ is independently selected from the group consisting of: hydrogen, and halogen. In a subclass of this class, each R⁷ is independently selected from the group consisting of: hydrogen, Cl and F. In another class of this embodiment, each R⁷ is hydrogen. In another class of this embodiment, R⁷ is halogen. In a subclass of this class, each R⁷ is independently selected from the group consisting of: Cl and F.

In another embodiment of the present invention, R⁸ is selected from the group consisting of: hydrogen, —C₁₋₁₀ alkyl, —C₂₋₁₀ alkenyl, and —C₃₋₁₀ cycloalkyl, wherein alkyl, alkenyl, and cycloalkyl are unsubstituted or substituted with one to three substituents independently selected from R^{a}. In a class of this embodiment, R⁸ is selected from the group consisting of: hydrogen, and —C₁₋₁₀ alkyl, wherein alkyl is unsubstituted or substituted with one to three substituents independently selected from R^{a}. In a subclass of this class, R⁸ is —C₁₋₁₀ alkyl, wherein alkyl is unsubstituted or substituted with one to three substituents independently selected from R^{a}. In another subclass of this class, R⁸ is hydrogen.

In another embodiment of the present invention, R⁹ and R¹⁰ are each independently selected from: hydrogen, and —C₁₋₄ alkyl, wherein alkyl is unsubstituted or substituted with one to five fluorines. In a class of this embodiment of the present invention, R⁹ and R¹⁰ are each —C₁₋₄ alkyl, wherein alkyl is unsubstituted or substituted with one to five fluorines. In another class of this embodiment, R⁹ and R¹⁰ are hydrogen.

In another embodiment of the present invention, each R^{a} is independently selected from the group consisting of —OR^{e}, —NR^{c}S(O)ₘR^{e}, halogen, —S(O)ₘR^{e}, —S(O)ₘNR^{c}R^{d}, —NR^{c}R^{d}, —C(O)R^{e}, —OC(O)R^{e}, oxo, —CO₂R^{e}, —CN, —C(O)NR^{c}R^{d}, —NR^{c}C(O)R^{e}, —NR^{c}C(O)OR^{e}, —NR^{c}C(O)NR^{c}R^{d}, —CF₃, —OCF₃, —OCHF₂ and C₂₋₆ cycloheteroalkyl. In a class of this embodiment, each R^{a} is independently selected from the group consisting of halogen, and —CN. In another class of this embodiment, each R^{a} is halogen. In a subclass of this class, R^{a} is Cl or F. In another subclass of this class, R^{a} is F. In another class of this embodiment, each R^{a} is —CN.

In another embodiment of the present invention, each R^{b} is independently selected from the group consisting of: R^{a}, —C₁₋₁₀ alkyl, and —C₃₋₆ cycloalkyl. In a class of this embodiment, each R^{b} is R^{a}. In another class of this embodiment, each R^{b} is independently selected from the group consisting of: —C₁₋₁₀ alkyl, and —C₃₋₆ cycloalkyl. In another class of this embodiment, each R^{b} is independently selected from the group consisting of: R^{a} and —C₁₋₁₀ alkyl. In a class of this embodiment, each R^{b} is independently selected from the group consisting of: halogen and —C₁₋₁₀ alkyl. In a subclass of this class, each R^{b} is independently selected from the group consisting of: F, Cl and CH₃. In a subclass of this class, each R^{b} is independently selected from the group consisting of: F and CH₃.

In another embodiment of the present invention, R^{c} and R^{d} are each independently selected from the group consisting of hydrogen, —C₁₋₁₀ alkyl, —C₂₋₁₀ alkenyl, —C₃₋₆ cycloalkyl, —C₃₋₆ cycloalkyl-C₁₋₁₀ alkyl-, C₂₋₁₀ cycloheteroalkyl, C₂₋₁₀ cycloheteroalkyl-C₁₋₁₀ alkyl-, aryl, heteroaryl, aryl-C₁₋₁₀ alkyl-, and heteroaryl-C₁₋₁₀ alkyl-, wherein when R^{c}C and R^{d} are other than hydrogen, each R^{c} and R^{d} is unsubstituted or substituted with one to three substituents independently selected from R^{h}. In a class of this embodiment, R^{c} and R^{d} are each independently selected from the group consisting of: hydrogen, and —C₁₋₁₀ alkyl, wherein alkyl is unsubstituted or substituted with one to three substituents independently selected from R^{h}. In another class of this embodiment, R^{c} and R^{d} are hydrogen. In another class of this embodiment, R^{c} and R^{d} are each —C₁₋₁₀ alkyl, wherein alkyl is unsubstituted or substituted with one to three substituents independently selected from R^{h}.

In another embodiment of the present invention, each R^{e} is independently selected from the group consisting of hydrogen, —C₁₋₁₀ alkyl, —C₂₋₁₀ alkenyl, —C₃₋₆ cycloalkyl, —C₃₋₆ cycloalkyl-C₁₋₁₀ alkyl-, C₂₋₁₀ cycloheteroalkyl, C₂₋₁₀ cycloheteroalkyl-C₁₋₁₀ alkyl-, aryl, heteroaryl, aryl-C₁₋₁₀ alkyl-, and heteroaryl-C₁₋₁₀ alkyl-, wherein, when R^{e} is not hydrogen, each R^{e} is unsubstituted or substituted with one to three substituents selected from R^{h}. In a class of this embodiment, each R^{e} is independently selected from the group consisting of: hydrogen, and —C₁₋₁₀ alkyl, wherein alkyl is unsubstituted or substituted with one to three substituents selected from. R^{h}. In a subclass of this class, each R^{e} is hydrogen. In another subclass of this class, each R^{e} is —C₁₋₁₀ alkyl, wherein alkyl is unsubstituted or substituted with one to three substituents selected from R^{h}.

In another embodiment of the present invention, each R^{g} is independently selected from: —C(O)R^{e} and —C₁₋₁₀ alkyl, wherein alkyl is unsubstituted or substituted with one to five fluorines. In a class of this embodiment, each R^{g} is —C₁₋₁₀ alkyl, wherein alkyl is unsubstituted or substituted with one to five fluorines.

In another embodiment of the present invention, each R^{h} is independently selected from the group consisting of: halogen, —C₁₋₁₀ alkyl, —O—C₁₋₄ alkyl, —S(O)ₘ—C₁₋₄ alkyl, —CN, —CF₃, —OCHF₂, and —OCF₃. In a class of this embodiment, each R^{h} is independently selected from the group consisting of halogen, and —C₁₋₁₀ alkyl.

In another embodiment of the present invention, m is 0.

In another embodiment of the present invention, m is 1 or 2. In a class of this embodiment, m is 1. In another class of this embodiment, in is 2.

In another embodiment of the present invention, n is 0 or 1.

In another embodiment of the present invention, n is 0, 1 or 2. In a class of this embodiment, n is 1. In another class of this embodiment, n is 2. In another class of this embodiment, n is 3.

In another embodiment of the present invention, there are provided compounds of structural formula II having the indicated R stereochemical configuration at the stereogenic carbon atom marked with an *:

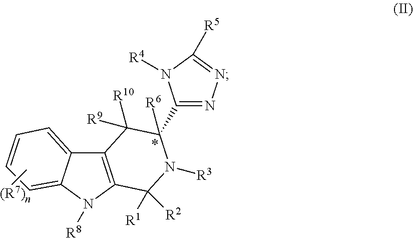

or a pharmaceutically acceptable salt thereof.

In another embodiment of the compounds of the present invention, R³, R⁴, R⁶, R⁸, R⁹, and R¹⁰ are each hydrogen. In a class of this embodiment, R⁵ is phenyl, unsubstituted or substituted with one to three substituents independently selected from R^{b}. In another class of this embodiment, R⁵ is phenyl, unsubstituted or substituted with one to three substituents independently selected from halogen, and R⁷ is hydrogen, halogen or CN. In another class of this embodiment, R⁵ is phenyl, unsubstituted or substituted with one to three fluorines, and R⁷ is hydrogen, F, Cl or CN.

In another embodiment of the compounds of the present invention, n is 0 or 1. In a class of this third embodiment R⁷ is hydrogen, halogen, or CN. In a subclass of this class, R⁷ is hydrogen, Cl or F. In a subclass of this subclass, R⁷ is hydrogen. In another subclass of this class, R⁷ is Cl. In another subclass of this class, R⁷ is F.

Illustrative, but nonlimiting examples, of the compounds of the present invention that are useful as antagonists of SSTR3 are the following beta-carbolines. Binding affinities for the SSTR3 receptor expressed as Kᵢ values are given below each structure.

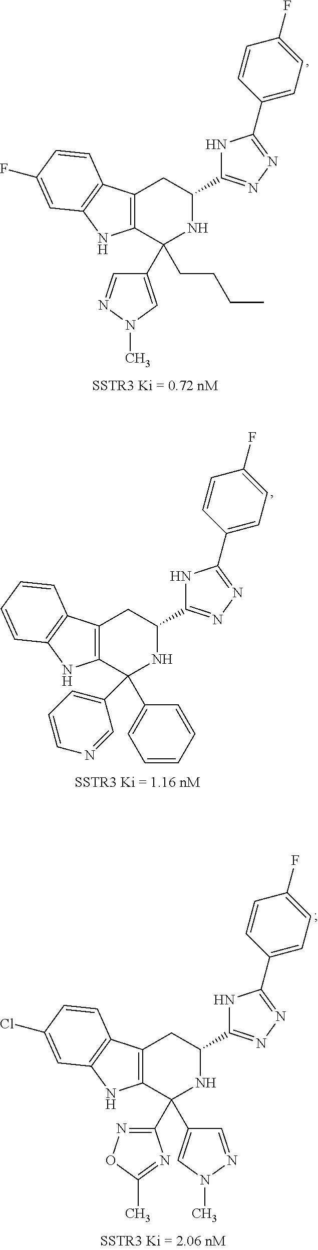

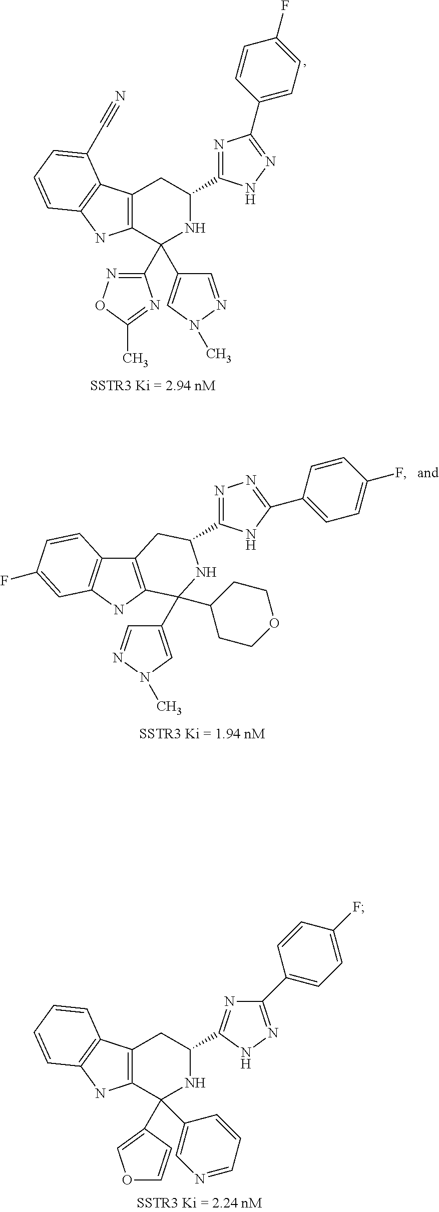

or a pharmaceutically acceptable salt thereof.

The SSTR3 as identified herein is a target for affecting insulin secretion and assessing beta-cell mass. Glucose stimulated insulin secretion was found to be stimulated by abrogating the expression of SSTR3 and through the use of an SSTR3 selective antagonist. An important physiological action of insulin is to decrease blood glucose levels. As disclosed in the present application, targeting the SSTR3 has different uses including therapeutic applications, diagnostic applications, and evaluation of potential therapeutics.

Somatostatin is a hormone that exerts a wide spectrum of biological effects mediated by a family of seven transmembrane (TM) domain O-protein-coupled receptors. (Lahlou et al., Ann. N.Y. Acad. Sci. 1014:121-131, 2004, Reisine et al., Endocrine Review 16 :427-442, 1995.) The predominant active forms of somatostatin are somatostatin-14 and somatostatin-28. Somatostatin-14 is a cyclic tetradecapeptide. Somatostatin-28 is an extended form of somatostatin-14.

Somatostatin subtype receptor 3 (SSTR3) is the third of five related G-protein receptor subtypes responding to somatostatin. The other receptors are the somatostatin subtype receptor 1 (SSTR1), somatostatin subtype receptor 2 (SSTR2), somatostatin subtype receptor 4 (SSTR4) and somatostatin subtype receptor 5 (SSTR5). The five distinct subtypes are encoded by separate genes segregated on different chromosomes. (Patel et al., Neuroendocrinol. 20:157-198, 1999). All five receptor subtypes bind somatostatin-14 and somatostatin-28, with low nanomolar affinity. The ligand binding domain for somatostatin is made up of residues in TMs III-VII with a potential contribution by the second extracellular loop. Somatostatin receptors are widely expressed in many tissues, frequently as multiple subtypes that coexist in the same cell.

The five different somatostatin receptors all functionally couple to inhibition of adenylate cyclase by a pertussin-toxin sensitive protein (G_{αi1-3}). (Lahlou et al., Ann. N.Y. Acad. Sci. 1014:121-131, 2004.) Somatostatin-induced inhibition of peptide secretion results mainly from a decrease in intracellular Ca²⁺.

Among the wide spectrum of somatostatin effects, several biological responses have been identified with different receptor subtypes selectivity. These include growth hormone (GH) secretion mediated by SSTR2 and SSTR5, insulin secretion mediated by SSTR1 and SSTR5, glucagon secretion mediated by SSTR2, and immune responses mediated by SSTR2. (Patel et al., Neuroendocrinol. 20:157-198, 1999; Crider et al., Expert Opin. Ther. Patents 13:1427-1441, 2003.)

Different somatostatin receptor sequences from different organisms are well known in the art. (See for example, Reisine et al., Endocrine Review 16 :427-442, 1995.) Human, rat, and murine SSTR3 sequences and encoding nucleic acid sequences are provided in SEQ ID NO: 3 (human SSTR3 cDNA gi|44890055|ref|NM|01051.21 CDS 526.1782); SEQ ID NO: 4 (human SSTR3AA gi|4557861|ref|NP_{—}001042.1|); SEQ ID NO: 5 (mouse SSTR3 cDNA gi|6678040|ref|NM_{—}009218.1| CDS1 . . . 1287); SEQ ID NO: 6 (mouse SSTR3 AA gi|6678041|ref|NP_{—}033244.1|); SEQ ID NO: 7 (rat SSTR3 cDNA gi|19424167|ref|NM_{—}133522.1| CDS 656 . . . 1942); SEQ ID NO: 8 (rat SSTR3A gi|19424168|ref|NP_{—}598206.1|). SSTR3 antagonists can be identified using SSTR3 and nucleic acid encoding for SSTR3.

Suitable assays include detecting compounds competing with a SSTR3 agonist for binding to SSTR3 and determining the functional effect of compounds on a SSTR3 cellular or physiologically relevant activity. SSTR3 cellular activities include cAMP inhibition, phospholipase C increase, tyrosine phsophatases increase, endothelial nitric oxide synthase (eNOS) decrease, K⁺ channel increase, Na⁺/H⁺ exchange decrease, and ERK decrease. (Lahlou et al., Ann. NY. Acad, Sci. 1014:121-131, 2004.) Functional activity can be determined using cell lines expressing SSTR3 and determining the effect of a compound on one or more SSTR3 activities (e.g., Poitout et al., J. Med. Chem. 44:29900-3000, 2001; Hocart et al., J. Med. Chem. 41:1146-1154, 1998).

SSTR3 binding assays can be performed by labeling somatostatin and determining the ability of a compound to inhibit somatostatin binding. (Poitout et al., J. Med. Chem. 44:29900-3000, 2001; Hocart et al., J. Med. Chem. 41:1146-1154, 1998.) Additional formats for measuring binding of a compound to a receptor are well-known in the art.

A physiologically relevant activity for SSTR3 inhibition is stimulating insulin secretion. Stimulation of insulin secretion can be evaluated in vitro or in vivo.

SSTR3 antagonists can be identified experimentally or based on available information. A variety of different SSTR3 antagonists are well known in the art. Examples of such antagonists include peptide antagonists, β-carboline derivatives, and a decahydroisoquinoline derivative. (Poitout et al., J. Med. Chem. 44:29900-3000, 2001, Hocart et al., J. Med. Chem. 41:1146-1154, 1998, Reubi et al., PNAS 97:13973-13978, 2000, Bänziger et al., Tetrahedron: Assymetry 14:3469-3477, 2003, Crider et al., Expert Opin. Ther. Patents 13:1427-1441, 2003, Troxler et al., International Publication No. WO 02/081471, International Publication Date Oct. 17, 2002).

Antagonists can be characterized based on their ability to bind to SSTR3 (Ki) and effect SSTR3 activity (IC₅₀), and to selectively bind to SSTR3 and selectively affect SSTR3 activity. Preferred antagonists strongly and selectively bind to SSTR3 and inhibit SSTR3 activity.

In different embodiments concerning SSTR3 binding, the antagonist has a Ki (nM) less than 100, preferably less than 50, more preferably less than 25 or more preferably less than 10. Ki can be measured as described by Poitout et al., J. Med. Chem. 44:29900-3000, 2001 and described herein.

A selective SSTR3 antagonist binds SSTR3 at least 10 times stronger than it binds SSTR1, SSTR2, SSTR4, and SSTR5. In different embodiments concerning selective SSTR3 binding, the antagonist binds to each of SSTR1, SSTR2, SSTR4, and SSTR5 with a Ki greater than 1000, or preferably greater than 2000 nM and/or binds SSTR3 at least 40 times, more preferably at least 100 times, or more preferably at least 500 times, greater than it binds to SSTR1, SSTR2, SSTR4, and SSTR5.

In different embodiments concerning SSTR3 activity, the antagonist has an IC₅₀ (nM) less than 500, preferably less than 100, more preferably less than 50, or more preferably less than 10 nM. IC₅₀ can be determined by measuring inhibition of somatostatin-14 induced reduction of cAMP accumulation due to forskolin (1 μM) in CHO-K1 cells expressing SSTR3, as described by Poitout et al., J. Med. Chem. 44:29900-3000, 2001.

Preferred antagonists have a preferred or more preferred Ki, a preferred or more preferred IC₅₀, and a preferred or more preferred selectivity. More preferred antagonists have a Ki (nM) less than 25; are at least 100 times selective for SSTR3 compared to SSTR1, SSTR2, SSTR4 and SSTR5; and have a IC₅₀ (nM) less than 50.

U.S. Pat. No. 6,586,445 discloses β-carboline derivatives as somatostatin receptor antagonists and sodium channel blockers denoted as being useful for the treatment of numerous diseases.

U.S. Pat. No. 6,861,430 also discloses β-carboline derivatives as SSTR3 antagonists for the treatment of depression, anxiety, and bipolar disorders.

Another set of examples are imidazolyl tetrahydro-β-carboline derivatives based on the compounds provided in Poitout et al., J. Med. Chem. 44:2990-3000, 2001.

Decahydroisoquinoline derivatives that are selective SSTR3 antagonists are disclosed in Bänziger et al., Tetrahedron: Assymetry 14:3469-3477, 2003.

“Alkyl”, as well as other groups having the prefix “alk”, such as alkoxy, alkanoyl, means carbon chains which may be linear or branched or combinations thereof. Examples of alkyl groups include methyl, ethyl, propyl, isopropyl, butyl, sec- and tert-butyl, pentyl, hexyl, heptyl, octyl, nonyl, and the like.

“Alkenyl” means carbon chains which contain at least one carbon-carbon double bond, and which may be linear or branched or combinations thereof. Examples of alkenyl include vinyl, allyl, isopropenyl, pentenyl, hexenyl, heptenyl, 1-propenyl, 2-butenyl, 2-methyl-2-butenyl, and the like.

“Alkynyl” means carbon chains which contain at least one carbon-carbon triple bond, and which may be linear or branched or combinations thereof. Examples of alkynyl include ethynyl, propargyl, 3-methyl-1-pentynyl, 2-heptynyl and the like.

“Cycloalkyl” means mono- or bicyclic or bridged saturated carbocyclic rings, each of which having from 3 to 10 carbon atoms. The term also includes monocyclic rings fused to an aryl group in which the point of attachment is on the non-aromatic portion. Examples of cycloalkyl include cyclopropyl, cyclobutyl, cyclopentyl, cyclohexyl, cycloheptyl, tetrahydronaphthyl, decahydronaphthyl, indanyl, and the like.

“Aryl” means mono- or bicyclic aromatic rings containing only carbon atoms. The term also includes aryl group fused to a monocyclic cycloalkyl or monocyclic cycloheteroalkyl group in which the point of attachment is on the aromatic portion. Examples of aryl include phenyl, naphthyl, indanyl, indenyl, tetrahydronaphthyl, 2,3-dihydrobenzofuranyl, dihydrobenzopyranyl, 1,4-benzodioxanyl, and the like.

“Heteroaryl” means an aromatic or partially aromatic heterocycle that contains at least one ring heteroatom selected from O, S and N. “Heteroaryl” thus includes heteroaryls fused to other kinds of rings, such as aryls, cycloalkyls and heterocycles that are not aromatic. Examples of heteroaryl groups include pyrrolyl, isoxazolyl, isothiazolyl, pyrazolyl, pyridyl (pyridinyl), oxazolyl, oxadiazolyl (in particular, 1,3,4-oxadiazol-2-yl and 1,2,4-oxadiazol-3-yl), thiadiazolyl, thiazolyl, imidazolyl, triazolyl, tetrazolyl, furyl, triazinyl, thienyl, pyrimidyl, benzisoxazolyl, benzoxazolyl, benzothiazolyl, benzothiadiazolyl, dihydrobenzofuranyl, indolinyl, pyridazinyl, indazolyl, isoindolyl, dihydrobenzothienyl, indolizinyl, cinnolinyl, phthalazinyl, quinazolinyl, naphthyridinyl, carbazolyl, 1,3-benzodioxolyl, benzo-1,4-dioxanyl, quinoxalinyl, purinyl, furazanyl, isobenzylfuranyl, benzimidazolyl, benzofuranyl, benzothienyl, quinolyl, indolyl, isoquinolyl, dibenzofuranyl, and the like. For heterocyclyl and heteroaryl groups, rings and ring systems containing from 3-15 atoms are included, forming 1-3 rings.

“Cycloheteroalkyl” and “C₂₋₁₀ Cycloheteroalkyl” mean mono- or bicyclic or bridged saturated rings containing at least one heteroatom selected from N, S and O, each of said ring having from 3 to 11 atoms in which the point of attachment may be carbon or nitrogen. The term also includes monocyclic heterocycle fused to an aryl or heteroaryl group in which the point of attachment is on the non-aromatic portion. Examples of “cycloheteroalkyl” include tetrahydropyranyl, tetrahydrofuranyl, pyrrolidinyl, piperidinyl, piperazinyl, dioxanyl, imidazolidinyl, 2,3-dihydrofuro(2,3-b)pyridyl, benzoxazinyl, benzoxazolinyl, 2-H-phthalazinyl, isoindolinyl, benzoxazepinyl, 5,6-dihydroimidazo[2,1-b]thiazolyl, tetrahydroquinolinyl, morpholinyl, tetrahydroisoquinolinyl, dihydroindolyl, and the like. The term also includes partially unsaturated monocyclic rings that are not aromatic, such as 2- or 4-pyridones attached through the nitrogen or N-substituted-(1H, 3H)-pyrimidine-2,4-diones (N-substituted uracils). The term also includes bridged rings such as 5-azabicyclo[2.2.1]heptyl, 2,5-diazabicyclo[2.2.1]heptyl, 2-azabicyclo[2.2.1]heptyl, 7-azabicyclo[2.2.1]heptyl, 2,5-diazabicyclo[2.2.2]octyl, 2-azabicyclo[2.2.2]octyl, and 3-azabicyclo[3.2.2]nonyl, and azabicyclo[2.2.1]heptanyl. The cycloheteroalkyl ring may be substituted on the ring carbons and/or the ring nitrogens.

“Halogen” includes fluorine, chlorine, bromine and iodine.

By “oxo” is meant the functional group “═O”, such as, for example, (1) “C═(O)”, that is a carbonyl group; (2) “S═(O)”, that is, a sulfoxide group; and (3) “N═(O)”, that is, an N-oxide group, such as pyridyl-N-oxide.

When any variable (e.g., R¹, R^{a}, etc.) occurs more than one time in any constituent or in formula I, its definition on each occurrence is independent of its definition at every other occurrence. Also, combinations of substituents and/or variables are permissible only if such combinations result in stable compounds.

Under standard nomenclature used throughout this disclosure, the terminal portion of the designated side chain is described first, followed by the adjacent functionality toward the point of attachment. For example, a C₁₋₅ alkylcarbonylamino C₁₋₆ alkyl substituent is equivalent to

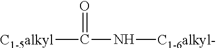

In choosing compounds of the present invention, one of ordinary skill in the art will recognize that the various substituents, i.e. R¹, R², etc., are to be chosen in conformity with well-known principles of chemical structure connectivity and stability.

The term “substituted” shall be deemed to include multiple degrees of substitution by a named substitutent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

### Optical Isomers—Diastereoisomers—Geometric Isomers—Tautomers:

Compounds of structural formula I may contain one or more asymmetric centers and can thus occur as racemates and racemic mixtures, single enantiomers, diastereoisomeric mixtures and individual diastereoisomers. The present invention is meant to comprehend all such isomeric forms of the compounds of structural formula I.

Compounds of structural formula I may be separated into their individual diastereoisomers by, for example, fractional crystallization from a suitable solvent, for example methanol or ethyl acetate or a mixture thereof, or via chiral chromatography using an optically active stationary phase. Absolute stereochemistry may be determined by X-ray crystallography of crystalline products or crystalline intermediates which are derivatized, if necessary, with a reagent containing an asymmetric center of known absolute configuration.

Alternatively, any stereoisomer of a compound of the general structural formula I may be obtained by stereospecific synthesis using optically pure starting materials or reagents of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereoisomeric mixture, followed by separation of the individual diastereoisomers by standard methods, such as fractional crystallization or chromatography, such as chiral chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Some of the compounds described herein contain olefinic double bonds, and unless specified otherwise, are meant to include both E and Z geometric isomers.

Some of the compounds described herein may exist as tautomers which have different points of attachment of hydrogen accompanied by one or more double bond shifts. For example, a ketone and its enol form are keto-enol tautomers. The individual tautomers as well as mixtures thereof are encompassed with compounds of the present invention. Examples of tautomers which are intended to be encompassed within the compounds of the present invention are illustrated below:

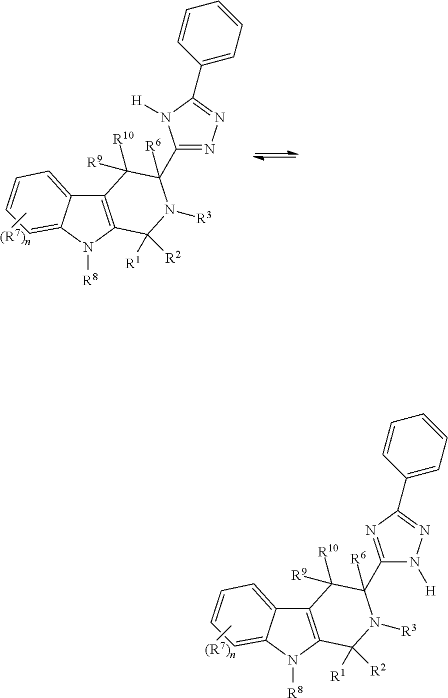

### Salts:

It will be understood that, as used herein, references to the compounds of structural formula I are meant to also include the pharmaceutically acceptable salts, and also salts that are not pharmaceutically acceptable when they are used as precursors to the free compounds or their pharmaceutically acceptable salts or in other synthetic manipulations.

The compounds of the present invention may be administered in the form of a pharmaceutically acceptable salt. The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, benzenesulfonate, benzoate, bicarbonate, bisulfate, bitartrate, borate, bromide, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

Also, in the case of a carboxylic acid (—COOH) or alcohol group being present in the compounds of the present invention, pharmaceutically acceptable esters of carboxylic acid derivatives, such as methyl, ethyl, or pivaloyloxymethyl, or acyl derivatives of alcohols, such as O-acetyl, O-pivaloyl, O-benzoyl, and O-aminoacyl, can be employed. Included are those esters and acyl groups known in the art for modifying the solubility or hydrolysis characteristics for use as sustained-release or prodrug formulations.

Solvates, and in particular, the hydrates of the compounds of structural formula I are included in the present invention as well.

Exemplifying the invention is the use of the compounds disclosed in the Examples and herein.

### Utilities:

The compounds described herein are potent and selective antagonists of the somatostatin subtype receptor 3 (SSTR3). The compounds are efficacious in the treatment of diseases that are modulated by SSTR3 ligands, which are generally antagonists. Many of these diseases are summarized below.

One or more of the following diseases may be treated by the administration of a therapeutically effective amount of a compound of Formula I, or a pharmaceutically acceptable salt thereof, to a patient in need of treatment. Also, the compounds of Formula I may be used for the manufacture of a medicament for treating one or more of these diseases:

(1) non-insulin dependent diabetes mellitus (Type 2 diabetes);

(2) hyperglycemia;

(3) Metabolic Syndrome;

(4) obesity;

(5) hypercholesterolemia;

(6) hypertriglyceridemia (elevated levels of triglyceride-rich-lipoproteins);

(7) mixed or diabetic dyslipidemia;

(8) low HDL cholesterol;

(9) high LDL cholesterol;

(10) hyperapoBlipoproteinemia; and

(11) atherosclerosis.

One embodiment of the uses of the compounds is directed to the treatment of one or more of the following diseases by administering a therapeutically effective amount to a patient in need of treatment. The compounds may be used for manufacturing a medicament for use in the treatment of one or more of these diseases:

(1) Type 2 diabetes;

(2) hyperglycemia;

(3) Metabolic Syndrome;

(4) obesity; and

(5) hypercholesterolemia.

The compounds are expected to be effective in lowering glucose and lipids in diabetic patients and in non-diabetic patients who have impaired glucose tolerance and/or are in a pre-diabetic condition. The compounds may ameliorate hyperinsulinemia, which often occurs in diabetic or pre-diabetic patients, by modulating the swings in the level of serum glucose that often occurs in these patients. The compounds may also be effective in treating or reducing insulin resistance. The compounds may be effective in treating or preventing gestational diabetes.

The compounds, compositions, and medicaments as described herein may also be effective in reducing the risks of adverse sequelae associated with metabolic syndrome, and in reducing the risk of developing atherosclerosis, delaying the onset of atherosclerosis, and/or reducing the risk of sequelae of atherosclerosis. Sequelae of atherosclerosis include angina, claudication, heart attack, stroke, and others.

By keeping hyperglycemia under control, the compounds may also be effective in delaying or preventing vascular restenosis and diabetic retinopathy.

The compounds of this invention may also have utility in improving or restoring β-cell function, so that they may be useful in treating type 1 diabetes or in delaying or preventing a patient with Type 2 diabetes from needing insulin therapy.

The compounds generally may be efficacious in treating one or more of the following diseases: (1) Type 2 diabetes (also known as non-insulin dependent diabetes mellitus, or NIDDM), (2) hyperglycemia, (3) impaired glucose tolerance, (4) insulin resistance, (5) obesity, (6) lipid disorders, (7) dyslipidemia, (8) hyperlipidemia, (9) hypertriglyceridemia, (10) hypercholesterolemia, (11) low HDL levels, (12) high LDL levels, (13) atherosclerosis and its sequelae; (14) vascular restenosis, (15) abdominal obesity, (16) retinopathy, (17) metabolic syndrome, (18) high blood pressure (hypertension), and (19) insulin resistance.

One aspect of the invention provides a method for the treatment and control of mixed or diabetic dyslipidemia, hypercholesterolemia, atherosclerosis, low HDL levels, high LDL levels, hyperlipidemia, and/or hypertriglyceridemia, which comprises administering to a patient in need of such treatment a therapeutically effective amount of a compound having formula I. The compound may be used alone or advantageously may be administered with a cholesterol biosynthesis inhibitor, particularly an HMG-CoA reductase inhibitor such as lovastatin, simvastatin, rosuvastatin, pravastatin, fluvastatin, atorvastatin, rivastatin, itavastatin, or ZD-4522. The compound may also be used advantageously in combination with other lipid lowering drugs such as cholesterol absorption inhibitors (for example stanol esters, sterol glycosides such as tiqueside, and azetidinones such as ezetimibe), ACAT inhibitors (such as avasimibe), CETP inhibitors (for example torcetrapib and those described in published applications WO2005/100298, WO2006/014413, and WO2006/014357), niacin and niacin receptor agonists, bile acid sequestrants, microsomal triglyceride transport inhibitors, and bile acid reuptake inhibitors. These combination treatments may be effective for the treatment or control of one or more related conditions selected from the group consisting of hypercholesterolemia, atherosclerosis, hyperlipidemia, hypertriglyceridemia, dyslipidemia, high LDL, and low HDL.

### Administration and Dose Ranges:

Any suitable route of administration may be employed for providing a mammal, especially a human, with an effective dose of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like. Preferably compounds of Formula I are administered orally.

The effective dosage of active ingredient employed may vary depending on the particular compound employed, the mode of administration, the condition being treated and the severity of the condition being treated. Such dosage may be ascertained readily by a person skilled in the art.

When treating or controlling diabetes mellitus and/or hyperglycemia or hypertriglyceridemia or other diseases for which compounds of Formula I are indicated, generally satisfactory results are obtained when the compounds of the present invention are administered at a daily dosage of from about 0.1 milligram to about 100 milligram per kilogram of animal body weight, preferably given as a single daily dose or in divided doses two to six times a day, or in sustained release form. For most large mammals, the total daily dosage is from about 1.0 milligrams to about 1000 milligrams. In the case of a 70 kg adult human, the total daily dose will generally be from about 1 milligram to about 500 milligrams. For a particularly potent compound, the dosage for an adult human may be as low as 0.1 mg. In some cases, the daily dose may be as high as one gm. The dosage regimen may be adjusted within this range or even outside of this range to provide the optimal therapeutic response.

Oral administration will usually be carried out using tablets or capsules. Examples of doses in tablets and capsules are 0.1 mg, 0.25 mg, 0.5 mg, 1 mg, 2 mg, 5 mg, 10 mg, 25 mg, 50 mg, 100 mg, 200 mg, 300 mg, 400 mg, 500 mg, and 750 mg. Other oral forms may also have the same or similar dosages.

### Compositions:

Another aspect of the present invention provides compositions which comprise a compound of Formula I and a pharmaceutically acceptable carrier. The compositions of the present invention comprise a compound of Formula I or a pharmaceutically acceptable salt as an active ingredient, as well as a pharmaceutically acceptable carrier and optionally other therapeutic ingredients. The term “pharmaceutically acceptable salts” refers to salts prepared from pharmaceutically acceptable non-toxic bases or acids including inorganic bases or acids and organic bases or acids. A composition may also comprise a prodrug, or a pharmaceutically acceptable salt thereof, if a prodrug is administered.

The compositions include compositions suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

In practical use, the compounds of Formula I can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions as oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form in which case solid pharmaceutical carriers are employed. If desired, tablets may be coated by standard aqueous or nonaqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally as, for example, liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

In some instances, depending on the solubility of the compound or salt being administered, it may be advantageous to formulate the compound or salt as a solution in an oil such as a triglyceride of one or more medium chain fatty acids, a lipophilic solvent such as triacetin, a hydrophilic solvent (e.g. propylene glycol), or a mixture of two or more of these, also optionally including one or more ionic or nonionic surfactants, such as sodium lauryl sulfate, polysorbate 80, polyethoxylated triglycerides, and mono and/or diglycerides of one or more medium chain fatty acids. Solutions containing surfactants (especially 2 or more surfactants) will form emulsions or microemulsions on contact with water. The compound may also be formulated in a water soluble polymer in which it has been dispersed as an amorphous phase by such methods as hot melt extrusion and spray drying, such polymers including hydroxylpropylmethylcellulose acetate (HPMCAS), hydroxylpropylmethyl cellulose (HPMCS), and polyvinylpyrrolidinones, including the homopolymer and copolymers.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

Compounds of formula I may also be administered parenterally. Solutions or suspensions of these active compounds can be prepared in water suitably mixed with a surfactant or mixture of surfactants such as hydroxypropylcellulose, polysorbate 80, and mono and diglycerides of medium and long chain fatty acids. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), suitable mixtures thereof, and vegetable oils.

### Combination Therapy:

Compounds of Formula I may be used in combination with other drugs that may also be useful in the treatment or amelioration of the diseases or conditions for which compounds of Formula I are useful. Such other drugs may be administered, by a route and in an amount commonly used therefor, contemporaneously or sequentially with a compound of Formula I. In the treatment of patients who have Type 2 diabetes, insulin resistance, obesity, metabolic syndrome, and co-morbidities that accompany these diseases, more than one drug is commonly administered. The compounds of this invention may generally be administered to a patient who is already taking one or more other drugs for these conditions. Often the compounds will be administered to a patient who is already being treated with one or more antidiabetic compound, such as metformin, sulfonylureas, and/or PPAR agonists, when the patient's glycemic levels are not adequately responding to treatment.

When a compound of Formula I is used contemporaneously with one or more other drugs, a pharmaceutical composition in unit dosage form containing such other drugs and the compound of Formula I is preferred. However, the combination therapy also includes therapies in which the compound of Formula I and one or more other drugs are administered on different overlapping schedules. It is also contemplated that when used in combination with one or more other active ingredients, the compound of the present invention and the other active ingredients may be used in lower doses than when each is used singly. Accordingly, the pharmaceutical compositions of the present invention include those that contain one or more other active ingredients, in addition to a compound of Formula I.

Examples of other active ingredients that may be administered in combination with a compound of Formula I, and either administered separately or in the same pharmaceutical composition, include, but are not limited to:

(a) PPAR gamma agonists and partial agonists, including both glitazones and non-glitazones (e.g. troglitazone, pioglitazone, englitazone, MCC-555, rosiglitazone, balaglitazone, netoglitazone, T-131, LY-300512, LY-818, and compounds disclosed in WO02/08188, WO2004/020408, and WO2004/020409.

(b) biguanides, such as metformin and phenformin;

(c) protein tyrosine phosphatase-1B (PTP-1B) inhibitors;

(d) dipeptidyl peptidase-IV (DPP-4) inhibitors, such as sitagliptin, saxagliptin, vildagliptin, and alogliptin;

(e) insulin or insulin mimetics;

(f) sulfonylureas such as tolbutamide, glimepiride, glipizide, and related materials;

(g) α-glucosidase inhibitors (such as acarbose);

(h) agents which improve a patient's lipid profile, such as (i) HMG-CoA reductase inhibitors (lovastatin, simvastatin, rosuvastatin, pravastatin, fluvastatin, atorvastatin, rivastatin, itavastatin, ZD-4522 and other statins), (ii) bile acid sequestrants (cholestyramine, colestipol, and dialkylaminoalkyl derivatives of a cross-linked dextran), (iii) niacin receptor agonists, nicotinyl alcohol, nicotinic acid, or a salt thereof, (iv) PPARα agonists, such as fenofibric acid derivatives (gemfibrozil, clofibrate, fenofibrate and bezafibrate), (v) cholesterol absorption inhibitors, such as ezetimibe, (vi) acyl CoA:cholesterol acyltransferase (ACAT) inhibitors, such as avasimibe, (vii) CETP inhibitors, such as torcetrapib, and (viii) phenolic antioxidants, such as probucol;

(i) PPARα/γ dual agonists, such as muraglitazar, tesaglitazar, farglitazar, and JT-501;

(j) PPARδ agonists, such as those disclosed in WO97/28149;

(k) anti-obesity compounds, such as fenfluramine, dexfenfluramine, phentiramine, subitramine, orlistat, neuropeptide Y Y5 inhibitors, MC4R agonists, cannabinoid receptor 1 (CB-1) antagonists/inverse agonists (e.g., rimonabant and taranabant), and 133 adrenergic receptor agonists;

(l) ileal bile acid transporter inhibitors;

(m) agents intended for use in inflammatory conditions, such as aspirin, non-steroidal anti-inflammatory drugs, glucocorticoids, azulfidine, and cyclooxygenase-2 (Cox-2) selective inhibitors;

(n) glucagon receptor antagonists;

(o) GLP-1;

(p) GIP-1;

(q) GLP-1 analogs and derivatives, such as exendins, (e.g., exenatide and liruglatide);

(r) 11β-hydroxysteroid dehydrogenase-1 (HSD-1) inhibitors;

(s) GPR40;

(t) GPR119; and

(u) SSTR5.

The above combinations include combinations of a compound of the present invention not only with one other active compound, but also with two or more other active compounds. Non-limiting examples include combinations of compounds having Formula I with two or more active compounds selected from biguanides, sulfonylureas, HMG-CoA reductase inhibitors, other PPAR agonists, PTP-1B inhibitors, DPP-4 inhibitors, and cannabinoid receptor 1 (CB1) inverse agonists/antagonists.

### Biological Assays

### Somatostatin Subtype Receptor 3 Production

SSTR3 can be produced using techniques well known in the art including those involving chemical synthesis and those involving recombinant production. (See e.g., Vincent, Peptide and Protein Drug Delivery, New York, N.Y., Decker, 1990; Current Protocols in Molecular Biology, John Wiley, 1987-2002, and Sambrook et al., Molecular Cloning, A Laboratory Manual, 2^{nd} Edition, Cold Spring Harbor Laboratory Press, 1989.)

Recombinant nucleic acid techniques for producing a protein involve introducing, or producing, a recombinant gene encoding the protein in a cell and expressing the protein. A purified protein can be obtained from cell. Alternatively, the activity of the protein in a cell or cell extract can be evaluated.

A recombinant gene contains nucleic acid encoding a protein along with regulatory elements for protein expression. The recombinant gene can be present in a cellular genome or can be part of an expression vector.

The regulatory elements that may be present as part of a recombinant gene include those naturally associated with the protein encoding sequence and exogenous regulatory elements not naturally associated with the protein encoding sequence. Exogenous regulatory elements such as an exogenous promoter can be useful for expressing a recombinant gene in a particular host or increasing the level of expression. Generally, the regulatory elements that are present in a recombinant gene include a transcriptional promoter, a ribosome binding site, a terminator, and an optionally present operator. A preferred element for processing in eukaryotic cells is a polyadenylation signal.

Expression of a recombinant gene in a cell is facilitated through the use of an expression vector. Preferably, an expression vector in addition to a recombinant gene also contains an origin of replication for autonomous replication in a host cell, a selectable marker, a limited number of useful restriction enzyme sites, and a potential for high copy number. Examples of expression vectors are cloning vectors, modified cloning vectors, specifically designed plasmids and viruses.

If desired, expression in a particular host can be enhanced through codon optimization. Codon optimization includes use of more preferred codons. Techniques for codon optimization in different hosts are well known in the art.

Enhancement of Glucose Dependent Insulin Secretion (GDIS) by SSTR3 antagonists in Isolated Mouse Islet Cells:

Pancreatic islets of Langerhans were isolated from the pancreas of normal C57BL/6J mice (Jackson Laboratory, Maine) by collagenase digestion and discontinuous Ficoll gradient separation, a modification of the original method of Lacy and Kostianovsky (Lacy et al., Diabetes 16:35-39, 1967). The islets were cultured overnight in RPMI 1640 medium (11 mM glucose) before GDIS assay.

To measure GDIS, islets were first preincubated for 30 minutes in the Krebs-Ringer bicarbonate (KRB) buffer with 2 mM glucose (in petri dishes). The KRB medium contains 143.5 mM Na⁺, 5.8 mM K⁺, 2.5 mM Ca²⁺, 1.2 mM Mg²⁺, 124.1 mM Cl⁻, 1.2 mM PO₄³⁻, 1.2 mM SO₄²⁺, 25 mM CO₃²⁻, 2 mg/mL bovine serum albumin (pH 7.4). The islets were then transferred to a 96-well plate (one islet/well) and incubated at 37° C. for 60 minutes in 200 μl of KRB buffer with 2 or 16 mM glucose, and other agents to be tested such as octreotide and a SST3 antagonist. (Zhou et al., J. Biol. Chem. 278:51316-51323, 2003.) Insulin was measured in aliquots of the incubation buffer by ELISA with a commercial kit (ALPCO Diagnostics, Windham, N.H.).

### SSTR Binding Assays:

The receptor-ligand binding assays of all 5 subtype of SSTRs were performed with membranes isolated from Chinese hamster ovary (CHO)-K1 cells stably expressing the cloned human somatostatin receptors in 96-well format as previous reported. (Yang et al. PNAS 95:10836-10841, 1998, Birzin et al. Anal. Biochem. 307:159-166, 2002.)

The stable cell lines for SSTR1-SSTR5 were developed by stably transfecting with DNA for all five SSTRs using Lipofectamine. Neomycin-resistant clones were selected and maintained in medium containing 400 μg/mL G418 (Rohrer et al. Science 282:737-740, 1998). Binding assays were performed using (3-¹²⁵I-Tyr11)-SRIF-14 as the radioligand (used at 0.1 nM) and The Packard Unifilter assay plate. The assay buffer consisted of 50 mM TrisHCl (pH 7.8) with 1 mM EGTA, 5 mM MgCl₂, leupeptin (10 μg/mL), pepstatin (10 μg/mL), bacitracin (200 μg/mL), and aprotinin (0.5 μg/mL). CHO-K1 cell membranes, radiolabeled somatostatin, and unlabeled test compounds were resuspended or diluted in this assay buffer. Unlabeled test compounds were examined over a range of concentrations from 0.01 nM to 10,000 nM. The Kᵢ values for compounds were determined as described by Cheng and Prusoff Biochem Pharmacol. 22:3099-3108 (1973).

Compounds of the present invention, particularly the compounds of Examples 1-19 and the Examples listed in Tables 2 and 3, exhibited Kᵢ values in the range of 100 nM to 0.1 nM against SSTR3 and exhibited Kᵢ values greater than 100 nM against SSTR1, SSTR2, SSTR4, and SSTR5 receptors.

### Functional Assay to Assess the Inhibition of SSTR3 Mediated Cyclic AMP Production:

The effects of compounds that bind to human and murine SSTR3 with various affinities on the functional activity of the receptor were assessed by measuring cAMP production in the presence of Forskolin (FSK) along or FSK plus SS-14 in SSTR3 expressing CHO cells. FSK acts to induce cAMP production in these cells by activating adenylate cyclases, whereas SS-14 suppresses cAMP production in the SSTR3 stable cells by binding to SSTR3 and the subsequent inhibition of adenylate cyclases via an alpha subunit of GTP-binding protein (Gαi).

To measure the agonism activity of the compounds, we pre-incubated the human or mouse SSTR3 stable CHO cells with the compounds for 15 min, followed by a one-hour incubation of the cells with 3.5 μM FSK (in the continuous presence of the compounds). The amount of cAMP produced during the incubation was quantified with the Lance cAMP assay kit (PerkinElmer, CA) according to the manufacturer's instruction. Majority of the compounds described in this application show no or little agonism activity. Therefore we used % Activation to reflect the agonism activity of each compound. The % Activation which was calculated with the following formula:

% Activation=[(FSK−Unknown)/(FSK−SS-14]×100

To measure the antagonism activity of the compounds, we pre-incubated the human or mouse SSTR3 stable CHO cells with the compounds for 15 min, followed by a one-hour incubation of the cells with a mixture of 3.5 μM FSK+100 nM SS-14 (in the continuous presence of the compounds). The amount of cAMP produced during the incubation was also quantified with the Lance cAMP assay. The antagonism activity of each compound was reflected both by % Inhibition (its maximum ability to block the action of SS-14) and an IC₅₀ value obtained by a eight-point titration. The % Inhibition of each compound was calculated using the following formula:

% Inhibition=[1−(unknown cAMP/FSK+SS-14 cAMP)]×100

In some case, 20% of human serum was included in the incubation buffer during the antagonism mode of the function assay to estimate the serum shift of the potency.

### Glucose Tolerance Test in Mice:

Male C57BL/6N mice (7-12 weeks of age) are housed 10 per cage and given access to noimal diet rodent chow and water ad libitum. Mice are randomly assigned to treatment groups and fasted 4 to 6 h. Baseline blood glucose concentrations are determined by glucometer from tail nick blood. Animals are then treated orally with vehicle (0.25% methylcellulose) or test compound. Blood glucose concentration is measured at a set time point after treatment (t=min) and mice are then challenged with dextrose intraperitoneally—(2-3 g/kg) or orally (3-5 g/kg). One group of vehicle-treated mice is challenged with saline as a negative control. Blood glucose levels are determined from tail bleeds taken at 20, 40, 60 minutes after dextrose challenge. The blood glucose excursion profile from t=0 to t=60 min is used to integrate an area under the curve (AUC) for each treatment. Percent inhibition values for each treatment are generated from the AUC data normalized to the saline-challenged controls. A similar assay may be performed in rats. Compounds of the present invention are active after an oral dose in the range of 0.1 to 100 mg/kg.

Abbreviations used in the following Schemes and Examples: aq. is aqueous; API-ES is atmospheric pressure ionization-electrospray (mass spectrum term); AcCN is acetonitrile; Boc is tert-butoxy carbonyl; d is day(s); DCM is dichloromethane; DEAD is diethyl azodicarboxylate; DIBAL is diisobutylaluminum hydride; DIPEA is N,N-diisopropylethylamine (Hunig's base); DMAP is 4-dimethylaminopyridine; DMF is N,N-dimethylformamide; DMSO is dimethylsulfoxide; EDC is 1-ethyl-3-(3-dimethylaminopropyl)-carbodiimide hydrochloride; EPA is ethylene polyacrylamide (a plastic); EtOAc is ethyl acetate; g is gram; h is hour(s); Hex is hexane; HOBt is 1-hydroxybenzotriazole; HPLC is high pressure liquid chromatography; HPLC/MS is high pressure liquid chromatography/mass spectrum; in vacuo means rotary evaporation under diminished pressure; IPA is isopropyl alcohol; IPAC or IPAc is isopropyl acetate; KHMDS is potassium hexamethyldisilazide; LC is liquid chromatography; LC-MS is liquid chromatography-mass spectrum; LDA is lithium diisopropylamide; M is molar; Me is methyl; MeOH is methanol; MHz is megahertz; mg is milligram; min is minute(s); mL is milliliter; mmol is millimole; MPLC is medium-pressure liquid chromatography; MS or ms is mass spectrum; MTBE is methyl tort-butyl ether; N is normal; NaHMDS is sodium hexamethyldisilazide; nm is nanometer; NMR is nuclear magnetic resonance; NMM is N-methylmorpholine; PyBOP is (benzotriazol-1-yloxy)tripyrrolidinophosphonium hexafluorophosphate; Rₜ is retention time; rt or RT is room temperature; sat. is saturated; TEA is triethylamine; TPA is trifluoroacetic acid; TFAA is trifluoroacetic acid anhydride; THF is tetrahydrofuran; and TLC or tlc is thin layer chromatography.

Several methods for preparing the compounds of this invention are illustrated in the following Schemes and Examples. Starting materials are either commercially available or made by known procedures in the literature or as illustrated. The present invention further provides processes for the preparation of compounds of structural formula I as defined above. In some cases the order of carrying out the foregoing reaction schemes may be varied to facilitate the reaction or to avoid unwanted reaction products. The following examples are provided for the purpose of illustration only and are not to be construed as limitations on the disclosed invention. All temperatures are degrees Celsius unless otherwise noted.

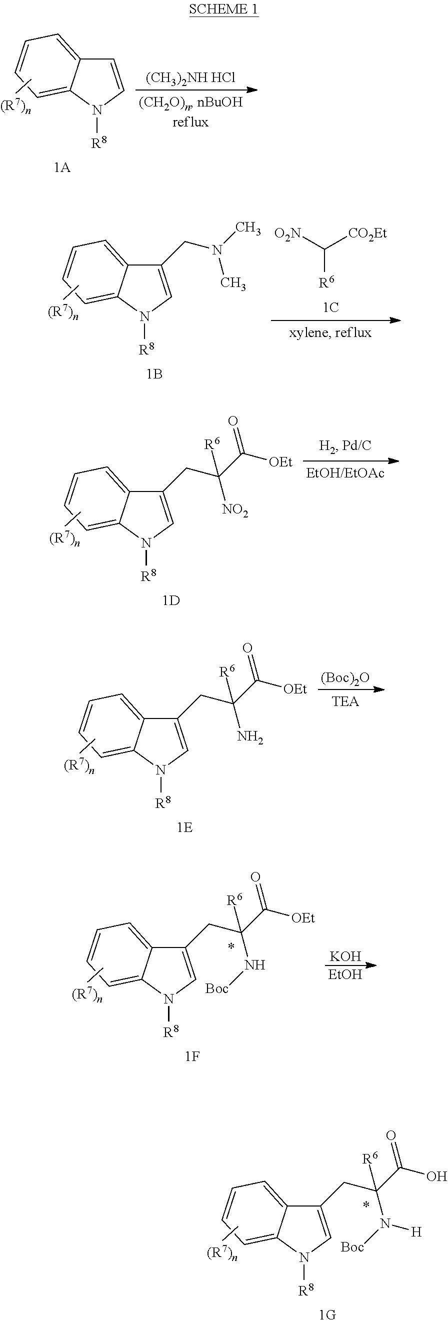

In Scheme 1, substituted indoles 1A are treated with dimethylamine and paraformaldehyde in a Mannich reaction to form 3-aminomethyl-indole 1B. Reaction of 1B with nitro ester 1C affords the 3-(indol-3-yl)-2-nitro-propionic acid, ethyl ester 1D, which is reduced to tryptophan derivative 1E. Acylation of the amine in 1E and subsequent hydrolysis of the resulting ester 1F affords the appropriately protected tryptophan derivative 1G. Separation of the isomers of 1F or 1G by chiral column chromatography yields the individual enantiomers at the * carbon.

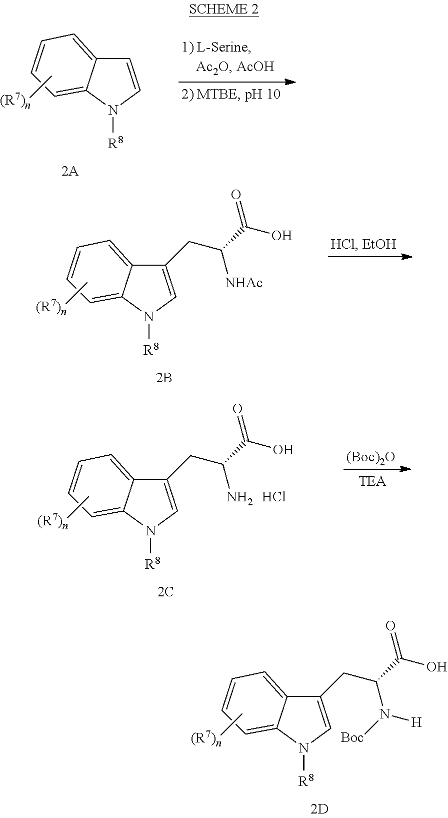

In Scheme 2, substituted indole 2A is reacted with L-serine in the presence of acetic anhydride and acetic acid to faun tryptophan 2B. Hydrolysis of the amide affords the desired enantiomer 2C. Protection of amine 2C with a Bac protecting group affords the Boc amine 2D.

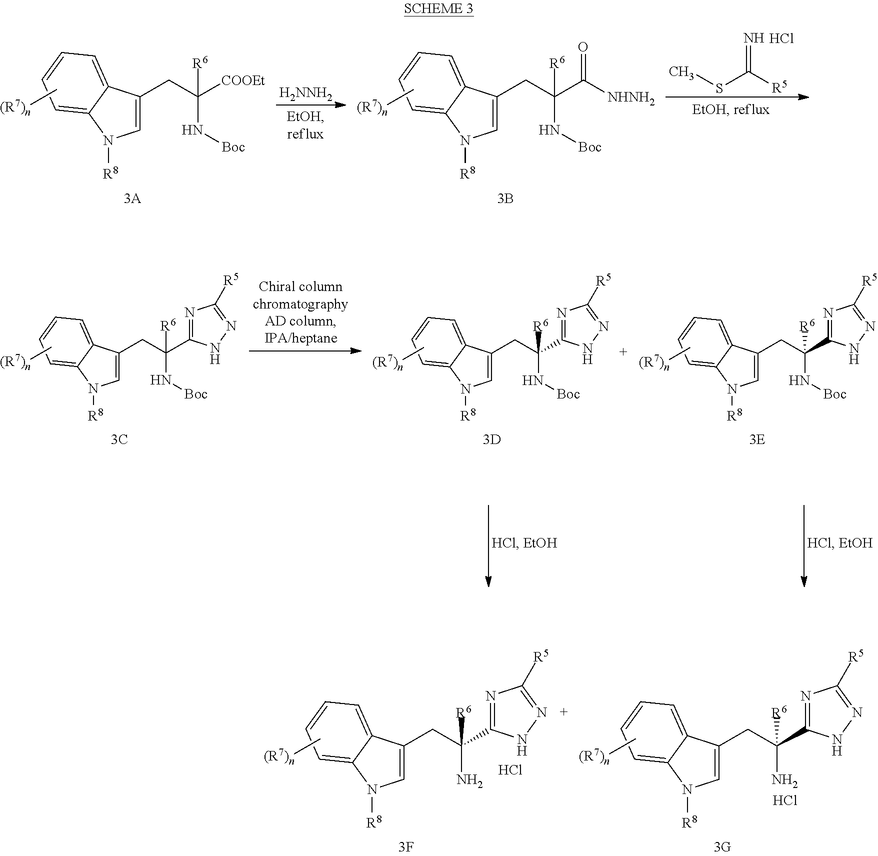

In Scheme 3, substituted tryptophan ester 3A (prepared according to methods outlined in Schemes 1 and 2) is reacted with hydrazine in refluxing ethanol to afford hydrazide 3B. This hydrazide is refluxed in ethanol with a thioimidate derivative to afford racemic triazole 3C, which is separated by chiral column chromatography to enantiomers 3D and 3E. The Boc group may be removed in the presence of strong acid to yield amines 3F and 3G.

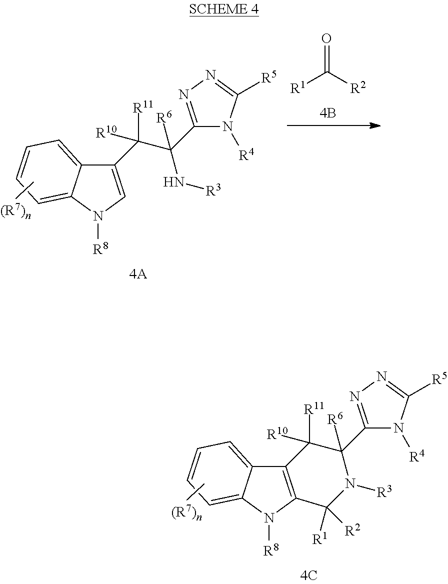

In Scheme 4, substituted triazolyl-tryptamine derivative 4A is reacted with an aldehyde or ketone 4B in a Pictet-Spengler cyclization to afford the desired β-carboline product 4C.

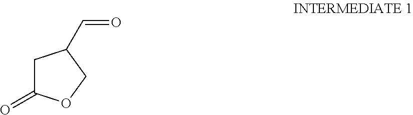

### Tetrahydrofuran-2-one-4-carboxaldehyde

Step A: 4-Hydroxymethyl-tetrahydrofuran-2-one. The title compound was prepared from tetrahydrofuran-2-one-4-carboxylic acid according to the methods described in the literature (Mori et al., Tetrahedron. 38:2919-2911, 1982.). ¹H NMR (500 MHz, CDCl₃): δ 5.02 (s, 1H), 4.42 (dd, 1H), 4.23 (dd, 1H), 3.67 (m, 2H), 2.78 (m, 1H), 2.62, (dd, 1H), 2.40, (dd, 1H).

Step B: Tetrahydrofuran-2-one-4-carboxaldehyde. To a solution of 4-hydroxymethyl-tetrahydrofuran-2-one (200 mg, 1.722 mmol) in CH₂Cl₂ (15 mL) was added Dess-Martin periodinane (804 mg, 1.895 mmol). The reaction was stirred at room temperature for 2.5 h. Sodium bicarbonate (1447 mg, 17.22 mmol) and water (2 mL) were added to the reaction. After stirring for 15 min, sodium thiosulfate (2723 mg, 17.22 mmol) was added, and the suspension was stirred for 15 additional min. The suspension was dried over sodium sulfate and filtered. The solid was washed with CH₂Cl₂. The organic layer was concentrated to a minimal volume to give the desired product. ¹H NMR (500 MHz, CDCl₃) showed an aldehyde singlet at δ 9.74 ppm. The crude product was used in subsequent reactions without further purification.

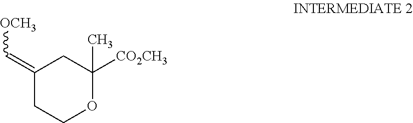

### 4-(Methoxymethylene)-2-methyl-tetrahydro-2H-pyran-2-carboxylic acid, methyl ester

Step A: 2-Methyl-2,3-dihydro-4H-pyran-4-one-2-carboxylic acid methyl ester. To a 100 mL one-neck round bottom flask was charged with Danishefsky's diene (5 g, 29.0 mmol) along with methyl pyvurate (3.11 g, 30.5 mmol) and toluene (50 mL). The mixture was stirred while a solution of ZnCl₂ (1M solution in ether, 2.90 mL, 2.90 mmol) was added dropwise over 5 min. The resulting reaction mixture was then stirred at room temperature for 18 h. The reaction was quenched by adding 0.1 N HCl (50 mL) and stirred at room temperature for 1 h. The organic layer was separated and the aqueous layer was extracted three times with ethyl acetate. The combined organic phases were washed with water, brine, dried over sodium sulfate, filtered and concentrated. The residue was purified by MPLC (120 g silica gel, 5 to 50% ethyl acetate in hexanes) to afford the product as a clear liquid. ¹H NMR (500 MHz, CDCl₃): δ 7.40 (d, 1H), 5.48 (d, 1H), 3.82 (s, 3H), 3.05 (d, 1H), 2.73 (d, 1H), 1.71, (s, 3H).

Step B: 2-Methyl-tetrapyran-4-one-2-carboxylic acid methyl ester. A suspension of 2-methyl-2,3-dihydro-4H-pyran-4-one-2-carboxylic acid, methyl este from Step A (3.54 g, 20.80 mmol) and Pd/C (2.214 g, 2.080 mmol) in methanol (50 mL) was attached to a H₂ balloon. The suspension was stirred at rt for 4 h. The reaction was filtered to remove the catalyst. The catalyst was washed was MeOH and filtrate concentrated to yield 2-methyl-tetrapyran-4-one-2-carboxylic acid, methyl ester. ¹H NMR (500 MHz, CDCl₃): δ 4.20 (m, 1H), 3.93 (m, 1H), 3.80 (s, 3H), 2.95 (d, 1H), 2.58 (m, 1H), 2.43 (m, 2H), 1.56 (s, 3H).

Step C: 4-(Methoxymethylene)-2-methyl-tetrahydro-2H-pyran-2-carboxylic acid, methyl ester. A suspension of (methoxymethyl)triphenylphosphonium chloride (7.71 g, 22.51 mmol) in THF (25 mL) was cooled to −20° C., and potassium tert-butoxide (18.00 mL, 18.00 mmol) in THF was added dropwise. After 10 min, a solution of 2-methyl-tetrapyran-4-one-2-carboxylic acid methyl ester from Step B (1.55 g, 9.00 mmol) in THF (15 mL) was added. The mixture was stirred for 30 min, then was warmed to RT and stirred for an additional hour. The mixture was cooled to −78° C. and quenched with saturated aqueous NH₄Cl. The mixture was extracted with EtOAc. The organic layers were washed with brine and dried over sodium sulfate. Silica gel column chromatography (hexane gradient to EtOAc) afforded 4-(methoxymethylene)-2-methyl-tetrahydro-2H-pyran-2-carboxylic acid methyl ester as a 1:1 mixture of double bond isomers. Characteristic peaks in ¹H NMR (500 MHz, CDCl₃): δ 5.93 (s, 1H) for one isomer, 5.90 (s, 1H) for the other isomer.

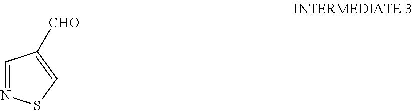

### Isothiazole-4-carboxaldehyde

Step A: N-Methoxy-N-methyl-isothiazole-4-carboxamide. A solution of isothiazole-4-carboxylic acid (1 g, 7.74 mmol) in CH₂Cl₂ (15 mL) and DMF (0.060 mL, 0.774 mmol) was cooled to 0° C., and oxalyl chloride (0.813 mL, 9.29 mmol) was added dropwise over 10 min. The reaction mixture was warmed to RT and stirred for 1 h. The resulting acid chloride solution was added to a cooled solution of N-methoxy-N-methyl-amine hydrochloride and K₂CO₃ (4.82 g, 34.8 mmol) in 10 mL water. The mixture was stirred at rt overnight and then extracted twice with EtOAc. The combined organic layers were washed with brine, dried over anhydrous Na₂SO₄, filtered, and concentrated to yield N-methoxy-N-methyl-isothiazole-4-carboxamide. ¹H NMR (400 MHz, CDCl₃): δ 9.25 (s, 1H), 8.93 (s, 1H), 3.66 (s, 3H), 3.36 (s, 3H).

Step B: Isothiazole-4-carboxaldehyde. Crude N-methoxy-N-methyl-isothiazole-4-carboxamide from Step A (0.91 g, 5.28 mmol) was dissolved in CH₂Cl₂ (15 mL) and cooled to −78° C. The solution was treated with DIBAL (15.85 mL, 15.85 mmol) and kept at −78° C. for 3 h. The reaction was quenched by dropwise addition of saturated aqueous NH₄Cl₂ (3 mL) at −78° C., warmed to rt and then kept cold overnight. The mixture was diluted with water and ether, treated with Rochelle's salt (6 g) and stirred at rt for 2 h. The organic layer was separated and the aqueous layer was extracted with ether. The combined organic layers were washed with brine, dried over anhydrous Na₂SO₄, and evaporated to afford isothiazole-4-carboxaldehyde, which was used without further purification. NMR (500 MHz, CDCl₃): δ 10.16 (s, 1H), 9.38 (s, 1H), 9.01 (s, 1H).

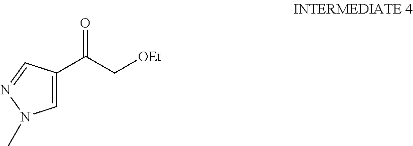

### 2-Ethoxy-1-(1-methyl-pyrazol-4-yl)-ethanone

Step A: N-Methoxy-N-methyl-2-ethoxyacetamide. A solution of ethoxyacetic acid (4.54 mL, 48.0 mmol) in CH₂Cl₂ (80 mL) and DMF (0.372 mL, 4.80 mmol) was cooled to 0° C. and oxalyl chloride (5.05 mL, 57.6 mmol) was added dropwise over 10 min. The reaction mixture was warmed up to rt and stirred for 1 h. The resulting acid chloride solution was added to a cooled solution of N-methoxy-N-methyl-amine hydrochloride and K₂CO₃ (29.9 g, 216 mmol) in 40 mL water. The mixture was stirred at rt overnight and extracted twice with ethyl acetate The combined organic layers were washed with brine, dried over anhydrous sodium sulfate, filtered and concentrated to afford crude N-methoxy-N-methyl-ethoxyacetamide, which was purified by silica gel column chromatography eluted with a CH₂Cl₂-to-acetone gradient. ¹H NMR (500 MHz, CDCl₃): δ 4.29 (s, 2H), 3.72 (s, 3H), 3.65 (q, 2H), 3.22 (s, 3H), 1.29 (t, 3H).

Step B: 2-Ethoxy-1-(1-methyl-pyrazol-4-yl)-ethanone. To a solution of 1-methyl-4-iodo-1H-pyrazole (3 g, 14.42 mmol) in THF (40 mL) was added isopropylmagnesium chloride (2.0M in THF, 8.00 mL, 16.01 mmol) at 0° C. The mixture was stirred at 0° C. for 1 h, cooled to −78° C. and N-methoxy-N-methyl-2-ethoxyacetamide (product of Step A, 3.18 g, 21.63 mmol) was added. The mixture was slowly warmed to rt over 1.5 h. The reaction was cooled to −78° C. and quenched by the dropwise addition of saturated aqueous NH₄Cl. The reaction was warmed to rt and stored in the cold overnight. The reaction was then diluted with cold 1N HCl, extracted four times with EtOAc, and the combined organic extracts were washed with brine, dried (Na₂SO₄) and concentrated. Silica gel chromatography eluting with a gradient of 50% EtOAc/hexanes to 100% EtOAc afforded 2-ethoxy-1-(1-methyl-pyrazol-4-yl)-ethanone. ¹H NMR (500 MHz, CDCl₃): δ 8.07 (s, 1H), 8.03 (s, 1H), 4.38 (s, 2H), 3.96 (s, 3H), 3.62 (q, 2H), 1.29 (t, 3H).

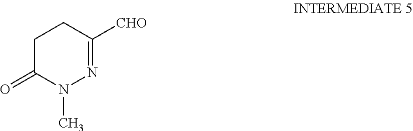

### 1-Methyl-6-oxo-1,4,5,6-tetrahydropyridazine-3-carboxaldehyde

Step A: 3-Hydroxymethyl-1-methyl-6-oxo-1,4,5,6-tetrahydropyridazine. 1-Methyl-6-oxo-1,4,5,6-tetrahydropyridazine-3-carboxylic acid (200 mg, 1.281 mmol) was dissolved in THF (2.0 mL). Triethylamine (0.179 mL, 1.281 mmol) was added, and the reaction was cooled in an ice bath. Ethyl chloroformate (0.168 mL, 1.281 mmol) was added in one portion. A precipitate formed and the mixture was stirred at the ice bath temperature for 15 minutes. NaBH₄ (121 mg, 3.2 mmol) in water (1.0 mL) was added, resulting in vigorous gas evolution. The ice bath was removed and the reaction was stirred at rt. for 2 hr. Some water was added and the mixture was extracted three times with CH₂Cl₂. The combined organic extracts were washed with brine (1×). The product was found to be water soluble. The aqueous layer was evaporated to dryness and triturated with CH₂Cl₂, with stirring for 15 min. The mixture was filtered and the solids were re-treated with CH₂Cl₂ with stirring for 10 min. The mixture was filtered, and the CH₂Cl₂ extracts were combined and evaporated to dryness. The resulting residue was dried under high vacuum at rt to afford the crude product as a colorless oil. The product was purified by flash chromatography on silica gel (1¼″×3¾″) by eluting with hexane-EtOAc-MeOH, 12:8:2) to afford 3-hydroxymethyl-1-methyl-6-oxo-1,4,5,6-tetrahydropyridazine as a colorless oil. LC-MS: single peak on UV curve at void volume (0.36 min); MS 100% peak is [M+H]+=143. ¹H-NMR (500 MHzMHZ, CDCl₃): δ CH₂—O (d4.31, s, 2H), N—CH₃ (d3.4, s, 3H), CH₂'s of ring (d2.54, m, 4H), OH+H₂O (d2.2, broad baseline peak, ˜2H).

Step B: 1-Methyl-6-oxo-1,4,5,6-tetrahydropyridazine-3-carboxaldehyde. Oxalyl chloride (382 μLμl, 4.36 mmol) was dissolved in CH₂Cl₂ (4.0 mL) and cooled to −70° C. DMSO (619 μLμl, 8.73 mmol) was added over a few minutes, resulting in vigorous gas evolution. The reaction mixture was stirred at −70° for 20 min, and a solution of 3-hydroxymethyl-1-methyl-6-oxo-1,4,5,6-tetrahydropyridazine (564 mg, 3.97 mmol) in CH₂Cl₂ (6 mL) was added over 5 min. A precipitate formed and the mixture was stirred at −70° C. for an additional 40 minutes. Triethylamine (2.76 mL, 19.84 mmol) was added, and the reaction warmed to rt. The mixture was then diluted with CH₂Cl₂ and a small amount of water was added along with some brine.

The layers were separated and the aqueous layer extracted twice with CH₂Cl₂ containing a small amount of MeOH. The combined extracts were dried over anhydrous MgSO₄, filtered, and concentrated by rotoevaporation. The resulting product was purified by flash chromatography on silica gel (1¼″×3½″) by eluting with hexane-EtOAc-MeOH (12:8:2) to afford 1-methyl-6-oxo-1,4,5,6-tetrahydropyridazine-3-carboxaldehyde as a pale yellow solid. LC-MS: single peak on UV curve at Rt=0.64 min. MS 100% peak is [M+H]+=141.

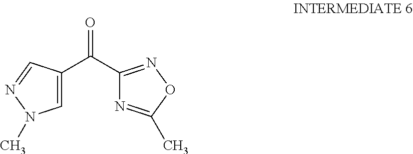

1-Methyl-pyrazol-4-yl5-methyl-1,2,4-triazol-3-yl ketone. To a solution of 1-methyl-4-iodo-1H-pyrazole (3 g, 14.42 mmol) in THF (40 mL) was added isopropylmagnesium chloride (2.0M in THF, 8.00 mL, 16.01 mmol) at 0° C. The mixture was stirred at 0° C. for 1 h, and then cooled to −78° C. N-methoxy-N-methyl-5-methyl-1,2,4-oxadiazole-3-carboxamide (prepared from the acid chloride of 5-methyl-1,2,4-oxadiazole-3-carboxylic acid and N-methoxy-N-methyl-amine hydrochloride according to the procedure described for the preparation of Intermediate 4, Step A) (3.21 g, 18.75 mmol) was added. The mixture was slowly warmed to rt over 1.5 h. The reaction was then cooled to −78° C. and quenched by the slow dropwise addition of a saturated solution of NH₄Cl. The resulting mixture was warmed to rt and then stored in a refrigerator overnight. The reaction was then diluted cold 1N aqueous HCl, and extracted four times with EtOAc. The combined organic layers were washed with brine and dried over anhydrous Na₂SO₄. The crude product was purified by silica gel chromatography by eluting with a gradient of 10% EtOAc in hexanes to 100% EtOAc to afford 1-methyl-pyrazol-4-yl 5-methyl-1,2,4-triazol-3-yl ketone. ¹H NMR (500 MHz, CDCl₃): δ 8.41 (s, 1H), 8.29 (s, 1H), 3.99 (s, 3H), 2.71 (s, 3H).

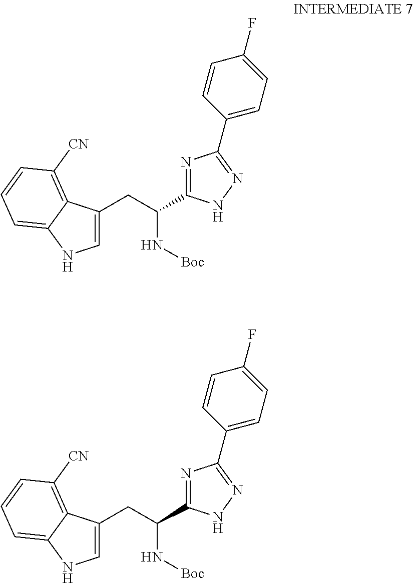

### Tert-butyl{(1R)-2-(4-cyano-1H-indol-3-yl)-1-[3-4(fluorophenyl)-1H-1,2,4-triazole-5-yl]ethyl}carbamate and tert-butyl{(1S)-2-(4-cyano-1H-indol-3-yl)-1-[3-4(fluorophenyl)-1H-1,2,4-triazole-5-yl}ethyl]carbamate

Step A: 1-(4-cyano-1H-indol-3-yl)-N,N-dimethylmethanamine. A 500 mL one neck round bottom flask was charged with 4-cyanoindole (5 g, 35.2 mmol), dimethylamine-hydrochloride (8.60 g, 106 mmol), paraformaldehyde (1.27 g, 42.2 mmol) and 1-butanol (100 mL). The resulting reaction mixture was stirred and heated to reflux for 1 hour. After cooling to room temperature, the mixture was diluted with ethyl acetate (100 mL) and washed with NaOH (1N, 120 mL). The organic layer was separated, and the aqueous layer was extracted with ethyl acetate (3×100 mL). The combined organic layers were washed with water, brine, dried over MgSO₄, filtered and concentrated to afford 1-(4-cyano-1H-indol-3-yl)-N,N-dimethylmethanamine as a solid. LC-MS: role 200 (M+H)⁺.

Step B: Ethyl 3-(4-cyano-1H-indol-3-yl)-2-nitropropanoate. A 500 mL three neck round bottom flask was charged with 1-(4-cyano-1H-indol-3-yl)-N,N-dimethylmethanamine (product of Step A, 7.01 g, 35.2 mmol), ethyl 2-nitroacetate (6.56 g, 49.3 mmol) and xylene (100 mL). The flask was equipped with a condenser, a nitrogen inlet and septum. The mixture was then heated to reflux with steady nitrogen flow through for 15 hours overnight. After cooling to room temperature, a solid product precipitated out and the solid was filtered and washed with ethyl acetate to afford ethyl 3-(4-cyano-1H-indol-3-yl)-2-nitropropanoate. LC-MS: m/e 288 (M+H)⁺. ¹HNMR (CD₃OD, 500 MHz) δ (ppm): 7.68 (1H, d, J=8.5 Hz), 7.47 (1H, d, J=7.5 Hz), 7.33 (1H, s) 7.24 (1H, t, J=7.5 Hz), 5.70 (1H, dd, J=9.5, 5.5 Hz), 4.24 (2H, q, J=6.0 Hz), 4.88 (2H, m), 1.21 (3H, t, J=6.0 Hz).

Step C: 4-Cyano-tryptophan ethyl ester. A 500 mL one neck round bottom flask was charged with ethyl 3-(4-cyano-1H-indol-3-yl)-2-nitropropanoate (product of step B, 8.33 g, 29.0 mmol), zinc (13.27 g, 203 mmol) and acetic acid (80 mL). The mixture was heated in an oil bath of 70° C. for 1 hour. After cooling to room temperature, the solvent was removed by rotary evaporation. The resulting residue was partitioned between ethyl acetate (100 mL) and saturated NaHCO₃ (100 mL). A large amount of Zn(OH)₂ formed, therefore the solid was filtered and washed with ethyl acetate before extraction. The organic layer was separated and the aqueous layer was extracted with ethyl acetate (3×). The combined organic layers were washed with brine, dried over MgSO₄, filtered and concentrated to afford 4-cyano-tryptophan ethyl ester. LC-MS: m/e 258 (M+H)⁺.

Step D: N-(tert-butoxycarbonyl)-4-cyano-tryptophan ethyl ester. A 500 mL one neck round bottom flask was charged with ethyl 4-cyano-tryptophanate (product of step C, 7.46 g, 29.0 mmol), THF (100 mL) and triethylamine (5.86 g, 58 mmol). Then Boc anhydride (6.33 g, 29.0 mmol) was added in one portion, and the reaction mixture was stirred further for 20 hours. The reaction was quenched with water (30 mL) and concentrated to give a residue. The residue was crystallized from ethyl acetate/hexanes (3:2) to afford the desired product. The mother liquid was concentrated and purified by MPLC to afford additional product N-(tert-butoxycarbonyl)-4-cyano-tryptophan ethyl ester. LC-MS: m/e 358 (M+H)⁺(1.13 min). ¹HNMR (CDCl₃, 500 MHz) δ (ppm): 7.68 (1H, d, J=8.5 Hz), 7.47 (1H, d, J=7.5 Hz), 7.33 (1H, s) 7.24 (1H, t, J=7.5 Hz), 5.70 (1H, dd, J=9.5, 5.5 Hz), 4.24 (2H, q, J=6.0 Hz), 4.88 (2H, m), 1.41 (9H, s), 1.21 (3H, t, J=6.0 Hz).

Step E: tert-butyl{1-[(4-cyano-1H-indol-3-yl)methyl]-2-hydrazino-2-oxoethyl}carbamate. A 100 mL one neck round bottom flask was charged with N-(tert-butoxycarbonyl)-4-cyano-tryptophan ethyl ester (product of step D, 3 g, 8.39 mmol), hydrazine (2.69 g, 84 mmol), and ethanol (10 mL). The mixture heated at reflux for 2 hours with stirring. The reaction mixture was then concentrated in vacuo and the resulting residue was azeotroped with toluene (2×) to afford crude tert-butyl{1-[(4-cyano-1H-indol-3-yl)methyl]-2-hydrazino-2-oxoethyl} carbamate. LC-MS: m/e 344 (M+H)⁺ (0.95 min).

Step F: tert-butyl{2-(4-cyano-1H-indol-3-yl)-1-[3-4(fluorophenyl)-1H-1,2,4-triazole-5-yl]ethyl}carbamate. A 100 mL one neck round bottom flask was charged with tert-butyl{1-[(4-cyano-1H-indol-3-yl)methyl]-2-hydrazino-2-oxoethyl}carbamate (product of step E, 1.5 g, 4.37 mmol), ethanol (10 mL), and 4-fluoro-benzenecarboximidothioic acid methyl ester (1.32 g, 4.46 mmol). The mixture was heated at reflux for 2 days. LC-MS showed loss of the Boc group. The reaction mixture was then concentrated and the residue was dissolved in methylene chloride, followed by treatment with Boc anhydride and triethyl amine. The mixture was stirred at room temperature for 2 hours. LC-MS showed the product was Boc protected. The reaction mixture was concentrated and partitioned between ethyl acetate (100 mL) and saturated NaHCO₃ (100 mL). The organic layer was separated and the aqueous layer was extracted with ethyl acetate (3×). The combined organic layers were washed with brine, dried, and concentrated. The resulting residue was purified by MPLC to afford tert-butyl{2-(4-cyano-1H-indol-3-yl)-1-[3-4(fluorophenyl)-1H-1,2,4-triazole-5-yl]ethyl}carbamate as a mixture of enantiomers. LC-MS: m/e 447 (M+H)⁺ (1.13 min).

Step G: tert-butyl{(1R)-2-(4-cyano-1H-indol-3-yl)-1-[3-4(fluorophenyl)-1H-1,2,4-triazole-5-yl]ethyl}carbamate and tert-butyl{(1S)-2-(4-cyano-1H-indol-3-yl)-1-[3-4(fluorophenyl)-1H-1,2,4-triazole-5-yl]ethyl}carbamate. The mixture of enantiomers of tert-butyl{2-(4-cyano-1H-indol-3-yl)-1-[3-4(fluorophenyl)-1H-1,2,4-triazole-5-yl]ethyl}carbamate (product of step F, 1 g, 2.24 mmol) was dissolved with isopropanol and resolved via a chiral AD column with 20% isopropanol in heptane. The faster eluting enantiomer, tert-butyl {(1R)-2-(4-cyano-1H-indol-3-yl)-1-[3-4(fluorophenyl)-1H-1,2,4-triazole-5-yl]ethyl}carbamate eluted at a retention time of 21.1 minutes. The slower eluting enantiomer, tert-butyl{(1s)-2-(4-cyano-1H-indol-3-yl)-1-[3-4(fluorophenyl)-1H-1,2,4-triazole-5-yl]ethyl}carbamate, eluted at a retention time of 31.1 minutes. LC-MS: m/e 447 (M+H)⁺(1.13 min). ¹HNMR (CD₃OD, 500 MHz) δ (ppm): 8.01 (2H, s), 7.63 (1H, d, J=10 Hz), 7.43 (1H, d, J=10 Hz) 7.19 (5H, m), 5.22 (1H, t,), 4.22 (1H, dd, J=8.0 Hz), 3.48 (1H, m), 1.36 (9H, s).

The Intermediates in Table 1 were prepared by the methods described for the preparation of Intermediate 7, replacing the cyano-indole or cyano-tryptophan with an appropriately substituted indole or tryptophan derivative. The individual enantiomers were separated by chiral chromatography as described in Intermediate 7, Step G.

TABLE 1

| Intermediate | Name | Structure |
|---|---|---|
| 8 | tert-butyl 2-(5- fluoro-1H-indo1-3- yl)-1-[3-(4- fluorophenyl)-1 H-1, 2, 4-triazol-5- yl]ethyl-carbamate | 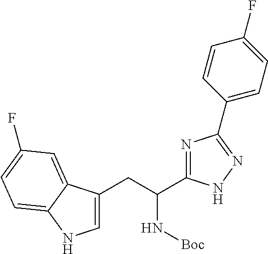 |
| 9 | tert-butyl 2-(5- chloro-1H-indo1-3- yl)-1-[3-(4- fluorophenyl)-1 H-1, 2, 4-triazol-5- yl]ethyl-carbamate | 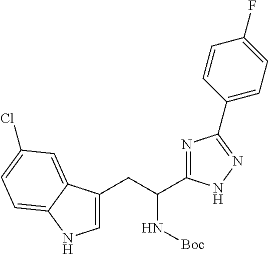 |
| 10 | tert-butyl 2-(6- fluoro-1H-indo1-3- yl)-1-[3-(4- fluorophenyl)-1 H-1, 2, 4-triazol-5- yl]ethyl-carbamate | 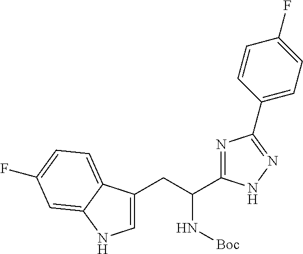 |
| 11 | tert-butyl 2-(6- chloro-1H-indol-3- yl)-1-[3-(4- fluorophenyl)-1 H-1, 2, 4-triazol-5- yl]ethyl-carbamate | 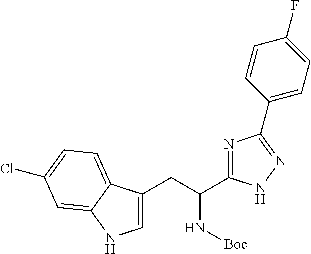 |

The compounds of the present invention are prepared according to the following examples, which are provided for the purpose of illustration only and are not to be construed as limitations on the disclosed invention.

### Example 1

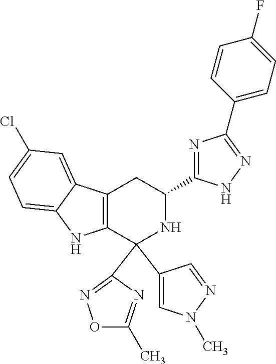

(3R)-6-chloro-3-[344 fluorophenyl)-1H-1,2,4-triazole-5-yl]-1-(5-methyl-1,2,4-oxadiazol-3-yl)-1-(1-methyl-1H-pyrazol-4-yl)-2,3,4,9-tetrahydro-1H-β-carboline A 25 mL sealed tube was charged with tert-butyl{(1R)-2-(5-chloro-1H-indol-3-yl)-1-[3-4(fluorophenyl)-1H-1,2,4-triazole-5-yl]ethyl}carbamate (Intermediate 9, 0.1 g, 0.219 mmol), methanol (1 mL) and HCl (16 M, 0.5 mL). The mixture was heated to 40° C. for 30 minutes, and then concentrated to give a residue. To the residue was added (5-methyl-1,2,4-oxadiazol-3-yl(1-methyl-1H-pyrazol-4-yl)methanone (0.05 g, 0.263 mmol), tetraethylorthosilicate (0.091 g, 0.439 mmol), and pyridine (0.5 mL). The mixture was degassed and refilled with nitrogen twice, then the cap was sealed. The reaction was heated to 95° C. overnight, then cooled and quenched with 10% NaCO₃. The resulting mixture was stirred for 30 minutes, and filtered. The resulting filtrate was partitioned between ethyl acetate and water. The aqueous layer was separated and extracted with ethyl acetate. The combined organic layers were dried over MgSO₄, filtered and concentrated to afford the crude product as a mixture of diastereomers. The crude product was purified by preparative TLC (ethyl acetate) to seperate the two diastereomers to give: Diastereomer D1 (the less polar diastereomer) (1s,3R)-6-chloro-3-[3-(4-fluorophenyl)-1H-1,2,4-triazole-5-yl]-1-(5-methyl-1,2,4-oxadiazol-3-yl)-1-(1-methyl-1H-pyrazol-4-yl)-2,3,4,9-tetrahydro-1H-β-carboline, characterized by LC-MS: m/e 530 (M+H)⁺(1.12 min), and ¹HNMR (CD₃OD, 500 MHz, D1) δ (ppm): 8.04 (2H, s), 7.63 (1H, s), 7.53 (1H, s), 7.46 (1H, d, J=9 Hz), 7.38 (1H, s), 7.22 (2H, t,), 7.02 (1H, d, J=9.0 Hz), 4.53 (1H, d, J=8.0 Hz), 3.86 (3H, s), 3.26 (1H, m), 3.15 (1H, m), 2.61 (3H, s); and Diastereomer D2 (the more polar diastereomer) (1R,3R)-6-chloro-3-[3-(4 fluorophenyl)-1H-1,2,4-triazole-5-yl]-1-(5-methyl-1,2,4-oxadiazol-3-yl)-1-(1-methyl-1H-pyrazol-4-yl)-2,3,4,9-tetrahydro-1H-β-carboline, characterized by: LC-MS: m/e 530 (M+H)⁺ (1.12 min) and ¹HNMR (CD₃OD, 500 MHz, D2) δ (ppm): 8.04 (2H, s), 7.53 (1H, s), 7.48 (1H, d, J=9 Hz) 7.44 (1H, s), 7.38 (1H, s), 7.22 (2H, t,), 7.02 (1H, d, J=9.0 Hz), 4.53 (1H, d, J=8.0 Hz), 3.86 (3H, s), 3.26 (1H, m), 3.15 (1H, m), 2.61 (3H, s).

The compounds of Examples 2-79 in Table 2 were prepared by the methods described for the preparation of the compound of Example 1, replacing the phenylimidazolyl chloro-indole derivative with an appropriately substituted indole or tryptophan derivative. Diastereomer 1 and Diastereomer 2 in Table 2 were separated from the corresponding mixture of diastereomers via chiral column chromatography. The retention times for the compounds and diastereomeric mixtures of compounds in Table 2 were determined by LC-MS (m/e) or electrospray ionization mass spec (M+H).

TABLE 2

| | | (m/e) | Retention | Single |
|---|---|---|---|---|
| Example | | or | Time | Compound |
| No. | Structure | (M + H) | (min) | or Mixture |
| 2 | 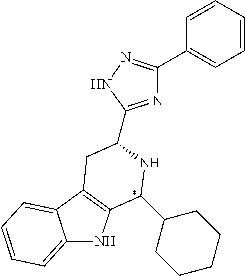 | 398 (M + H) | not available | Single diastereomer at * carbon |
| 3 | 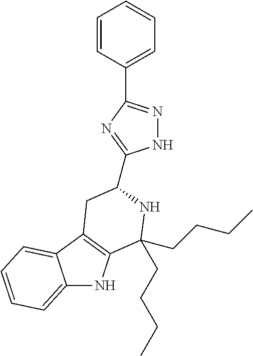 | 428 (M + H) | not available | Single enantiomer |
| 4 | 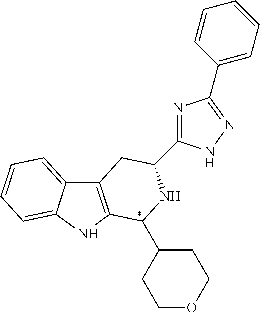 | 400 (m/e) | 2.55 | Single diastereomer at * carbon |
| 5 | 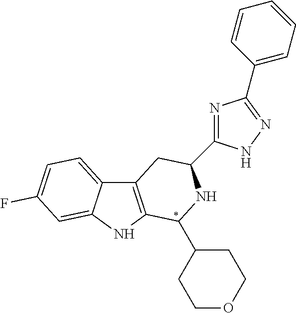 | 418 | 2.57 | Single diastereomer at * carbon |
| 6 | 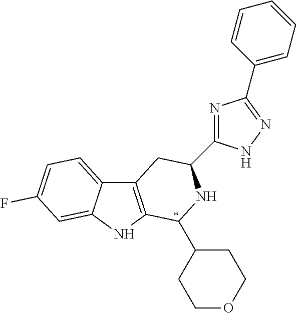 | 418 | 2.57 | Single diastereomer at * carbon |
| 7 | 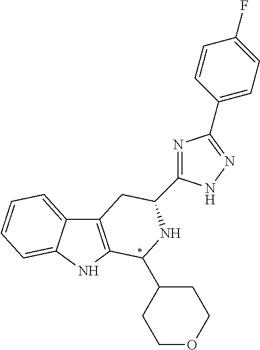 | 418 (m/e) | 2.47 | Single diastereomer at * carbon |
| 8 | 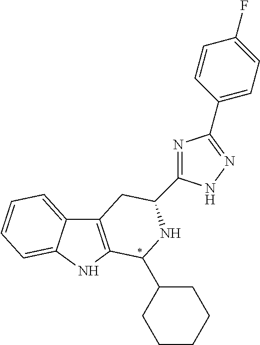 | 416 (m/e) | 2.84 | Single diastereomer at * carbon |
| 9 | 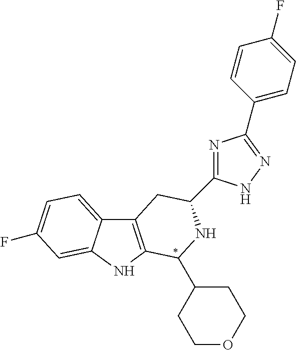 | 436 (m/e) | 1.04 | Single diastereomer at * carbon |
| 10 | 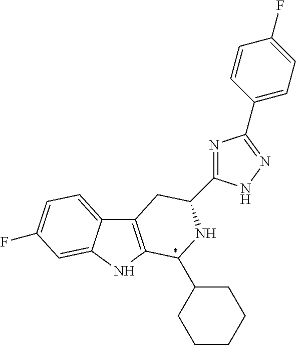 | 434 (m/e) | 1.09 | Single diastereomer at * carbon |
| 11 | 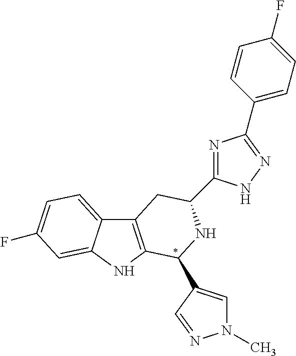 | 432 (m/e) | 1.02 | Diastereomer 1 at * carbon |
| 12 | 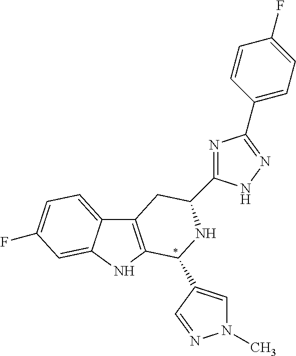 | 432 (m/e) | 1.03 | Diastereomer 2 at * carbon |
| 13 | 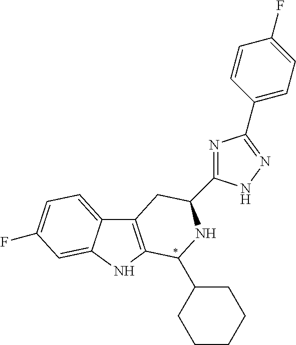 | 434 (m/e) | 1.09 | Single diastereomer at * carbon |
| 14 | 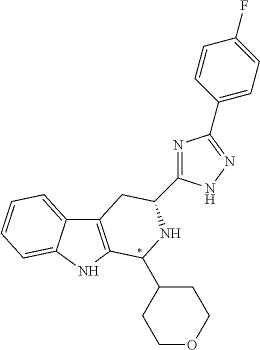 | 418 (m/e) | 1.03 | Single diastereomer at * carbon |
| 15 | 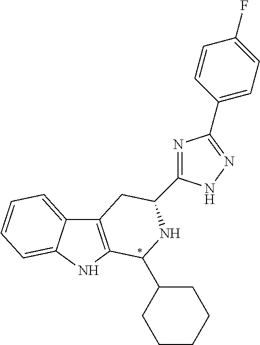 | 416 (m/e) | 1.09 | Single diastereomer at * carbon |
| 16 | 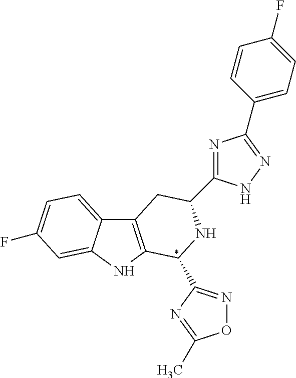 | 434 (m/e) | 1.07 | Diastereomer 1 at * carbon |
| 17 | 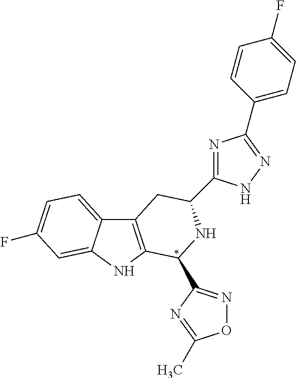 | 434 (m/e) | 1.07 | Diastereomer 2 at * carbon |
| 18 | 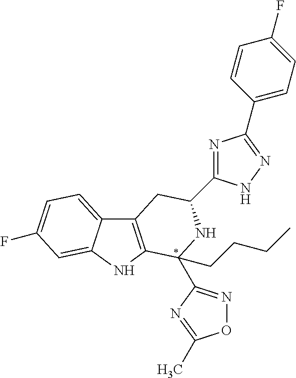 | 490 (m/e) | 1.17, 1.24 | Mixture of diastereomers at * carbon |
| 19 | 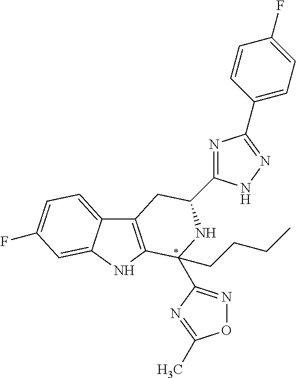 | 490 (m/e) | 1.14 | Diastereomer 1 at * carbon |
| 20 | 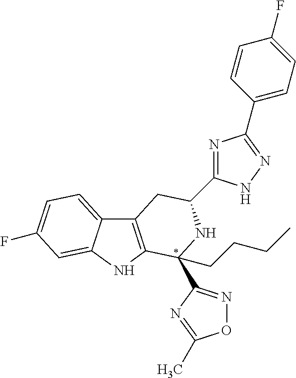 | 490 (m/e) | 1.15 | Diastereomer 2 at * carbon |
| 21 | 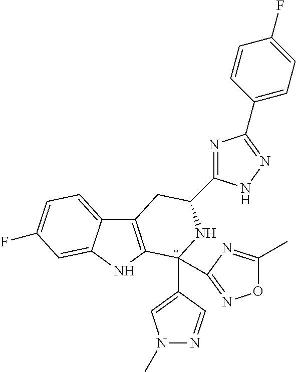 | 514 (m/e) | 1.09 | Mixture of Diastereomers at * carbon |
| 22 | 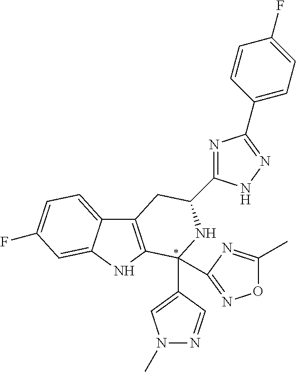 | 514 (m/e) | 1.09 | Diastereomer 1 at * carbon |
| 23 | 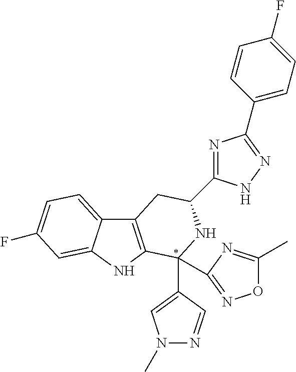 | 514 (m/e) | 1.09 | Diastereomer 2 at * carbon |
| 24 | 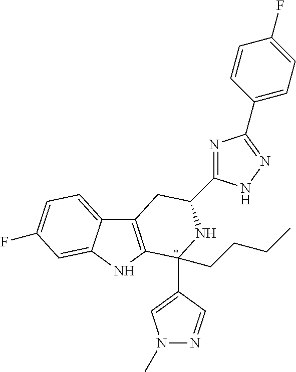 | 488 (m/e) | 1.05, 1.07 | Mixture of Diastereomers at * carbon |
| 25 | 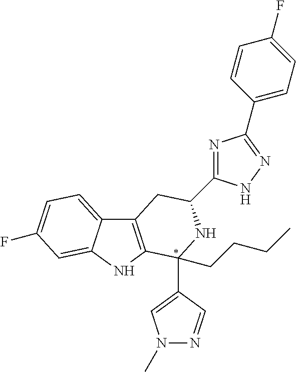 | 488 (m/e) | 1.08 | Diastereomer 1 at * carbon |
| 26 | 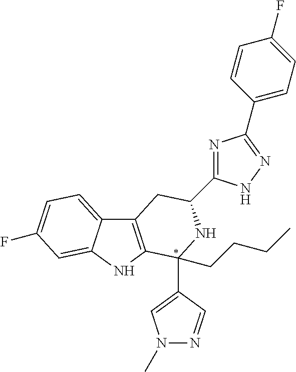 | 488 (m/e) | 1.05 | Diastereomer 2 at * carbon |
| 27 | 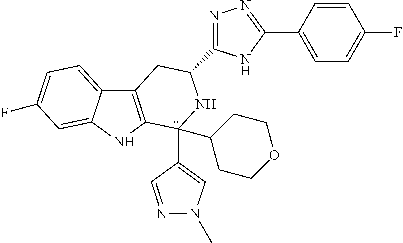 | 516 (m/e) | 2.70 | Diastereomer 1 at * carbon |
| 28 | 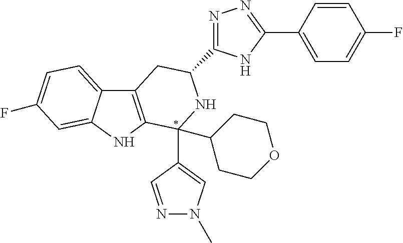 | 516 (m/e) | 2.42 | Diastereomer 2 at * carbon |
| 29 | 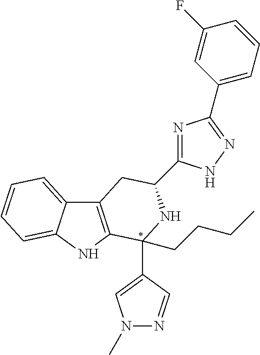 | 470 (m/e) | 1.08 | Diastereomer 1 at * carbon |
| 30 | 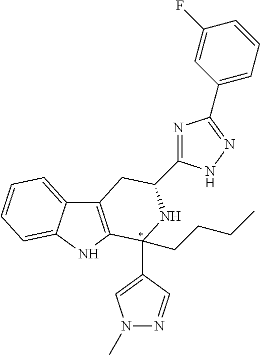 | 470 (m/e) | 1.05 | Diastereomer 2 at * carbon |
| 31 | 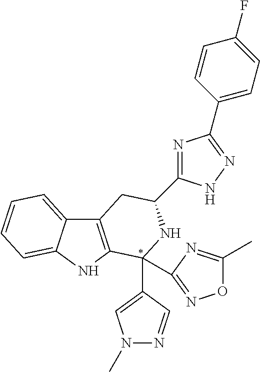 | 496 (m/e) | 1.08 | Diastereomer 1 at * carbon |
| 32 | 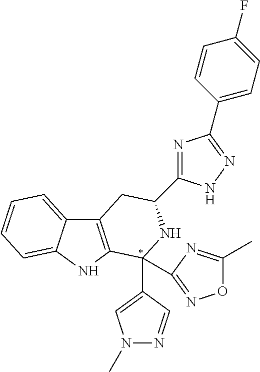 | 496 (m/e) | 1.07 | Diastereomer 2 at * carbon |
| 33 | 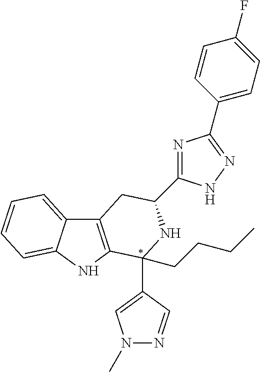 | 470 (m/e) | 1.07 | Diastereomer 1 at * carbon |
| 34 | 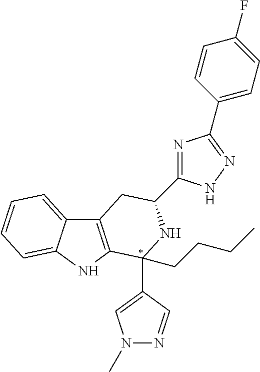 | 470 (m/e) | 1.03 | Diastereomer 2 at * carbon |
| 35 | 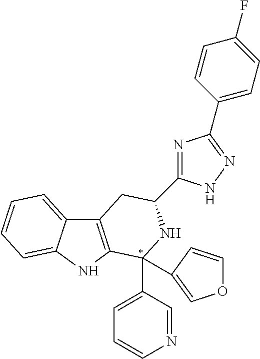 | 477 (m/e) | 1.07 | Mixture of diastereomers at * carbon |
| 36 | 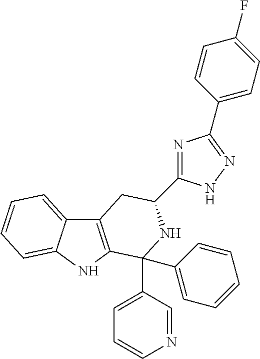 | 487 (m/e) | 1.09 | Single enantiomer |
| 37 | 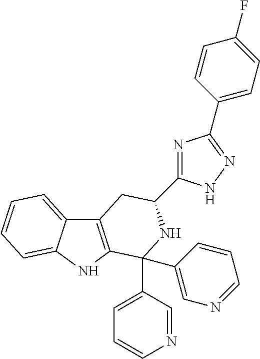 | 488 (m/e) | 1.02 | Single enantiomer |
| 38 | 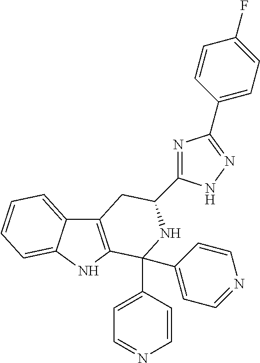 | 488 (m/e) | 1.00 | Single enantiomer |
| 39 | 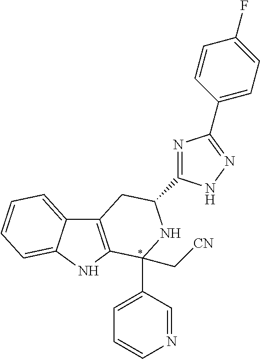 | 450 (m/e) | 1.04 | Mixture of diastereomers at * carbon |
| 40 | 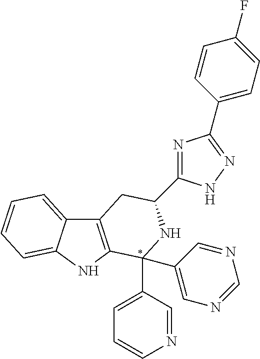 | 489 (m/e) | 1.02 | Diastereomer 1 at * carbon |
| 41 | 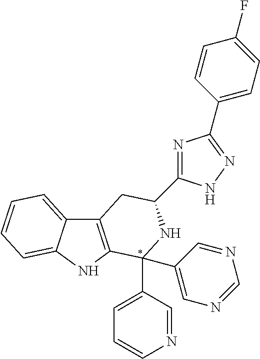 | 489 (m/e) | 1.04 | Diastereomer 2 at * carbon |
| 42 | 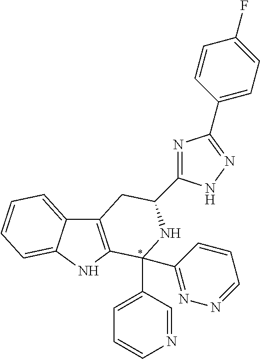 | 489 (m/e) | 1.00, 1.03 | Mixture of Diastereomers at * carbon |
| 43 | 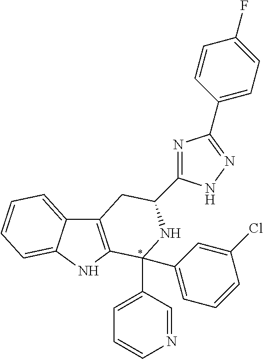 | 521 (m/e) | 1.09 | Mixture of diastereomers at * carbon |
| 44 | 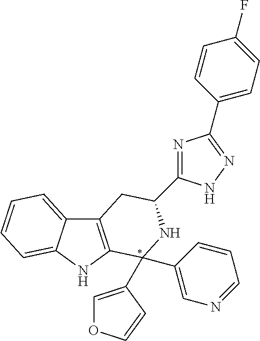 | 477 (m/e) | 1.06 | Diastereomer 1 at * carbon |
| 45 | 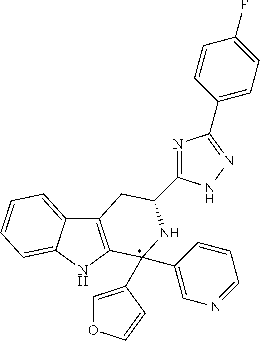 | 477 (m/e) | 1.06 | Diastereomer 2 at * carbon |
| 46 | 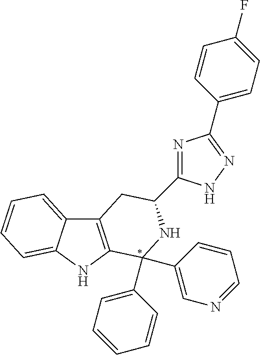 | 487 (m/e) | 1.07 | Diastereomer 1 at * carbon |
| 47 | 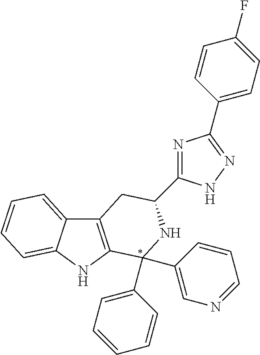 | 487 (m/e) | 1.09 | Diastereomer 2 at * carbon |
| 48 | 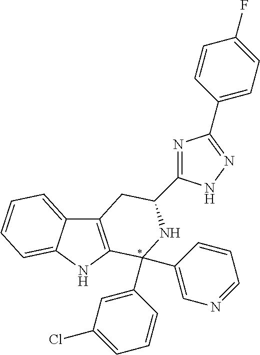 | 521 (m/e) | 1.10 | Diastereomer 1 at * carbon |
| 49 | 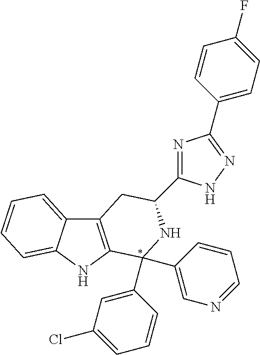 | 521 (m/e) | 1.12 | Diastereomer 2 at * carbon |
| 50 | 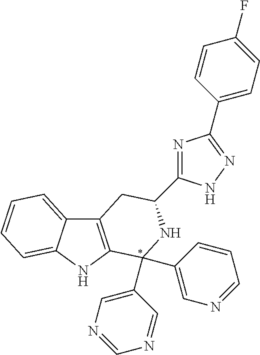 | 489 (m/e) | 1.03 | Diastereomer 1 at * carbon |
| 51 | 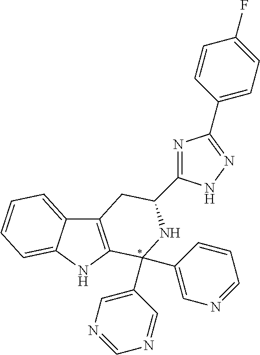 | 489 (m/e) | 1.03 | Diastereomer 2 at * carbon |
| 52 | 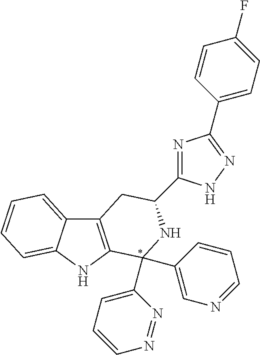 | 489 (m/e) | 1.02 | Diastereomer 1 at * carbon |
| 53 | 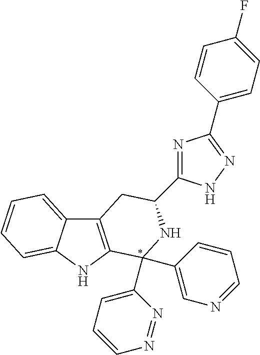 | 489 (m/e) | 1.03 | Diastereomer 2 at * carbon |
| 54 | 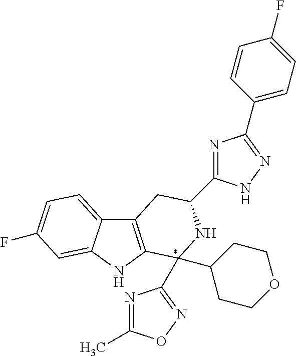 | 518 (m/e) | 1.14 | Mixture of Diastereomers at * carbon |
| 55 | 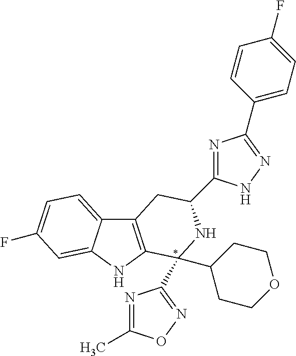 | 518 (m/e) | 1.14 | Diastereomer 1 at * carbon |
| 56 | 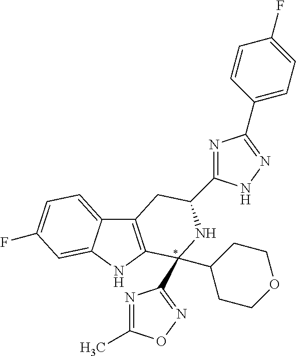 | 518 (m/e) | 1.15 | Diastereomer 2 at * carbon |
| 57 | 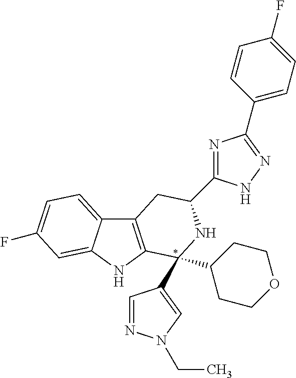 | 530 (m/e) | 1.07 | Diastereomer 1 at * carbon |
| 58 | 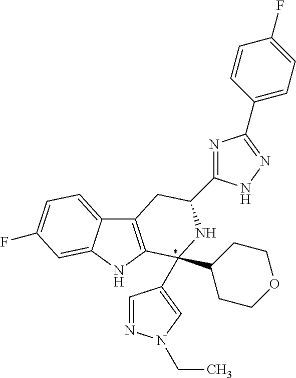 | 530 (m/e) | 1.03 | Diastereomer 2 at * carbon |
| 59 | 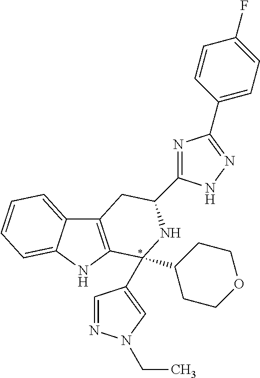 | 512 (m/e) | 1.06 | Diastereomer 1 at * carbon |
| 60 | 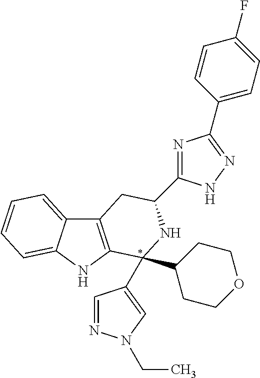 | 512 (m/e) | 1.00 | Diastereomer 2 at * carbon |
| 61 | 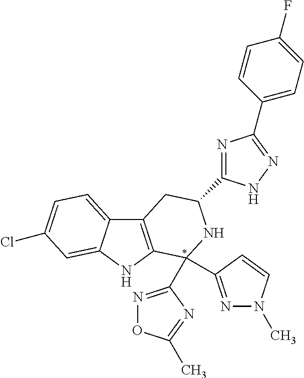 | 530 (m/e) | 1.12 | Diastereomer 1 at * carbon |
| 62 | 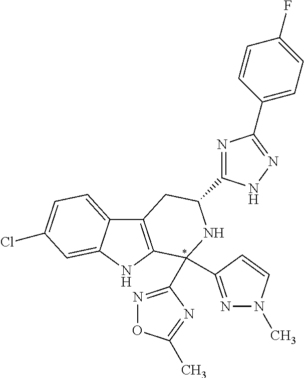 | 530 (m/e) | 1.12 | Diastereomer 2 at * carbon |
| 63 | 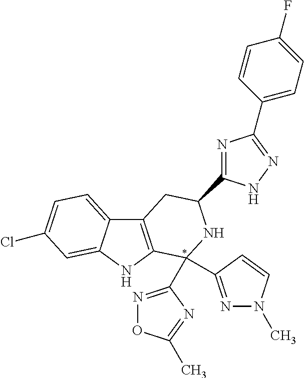 | 530 (m/e) | 1.14 | Diastereomer 1 at * carbon |
| 64 | 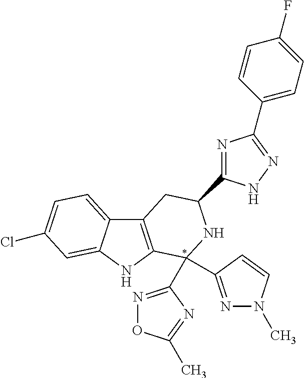 | 530 (m/e) | 1.14 | Diastereomer 2 at * carbon |
| 65 | 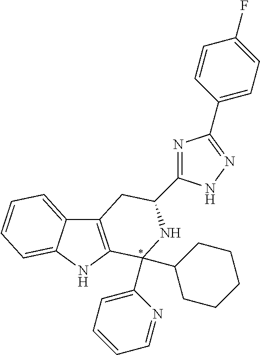 | 493 (m/e) | 1.11 | Mixture of Diastereomers at * carbon |
| 66 | 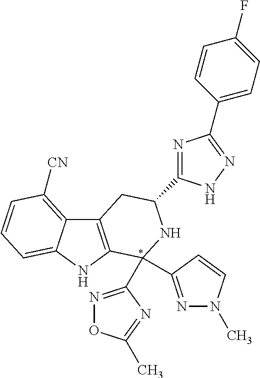 | 521.3 (m/e) | 1.11 | Mixture of Diastereomers at * carbon |
| 67 | 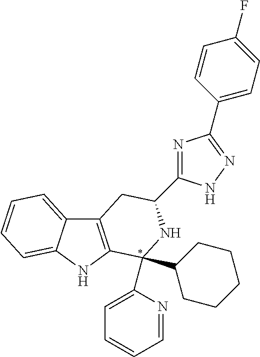 | 493 (m/e) | 1.11 | Diastereomer 1 at * carbon |
| 68 | 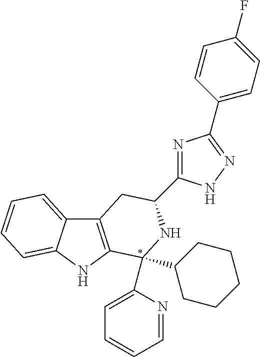 | 493 (m/e) | 1.10 | Diastereomer 2 at * carbon |
| 69 | 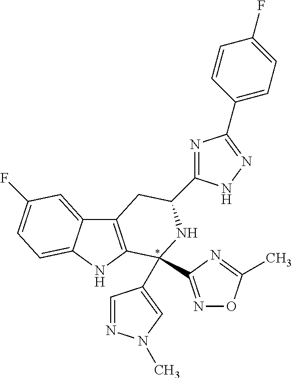 | 514 (m/e) | 1.09 | Diastereomer 1 at * carbon |
| 70 | 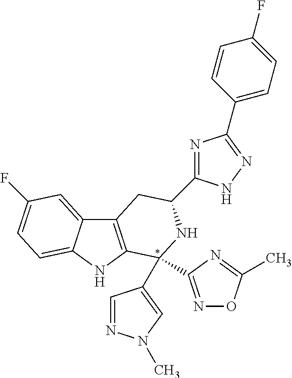 | 514 (m/e) | 1.09 | Diastereomer 2 at * carbon |
| 71 | 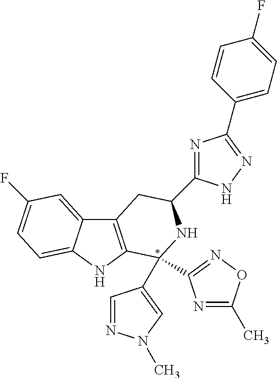 | 514 (m/e) | 1.09 | Diastereomer 1 at * carbon |
| 72 | 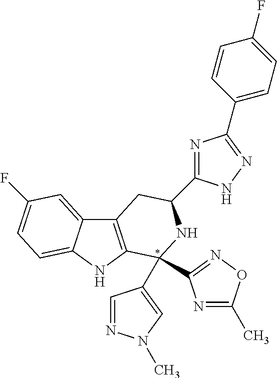 | 514 (m/e) | 1.09 | Diastereomer 2 at * carbon |
| 73 | 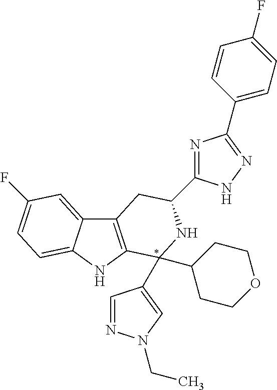 | 530 (m/e) | 1.07, 1.04 | Mixture of Diastereomers at * carbon |
| 74 | 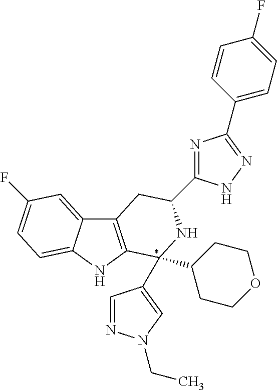 | 530 (m/e) | 1.06 | Diastereomer 1 at * carbon |
| 75 | 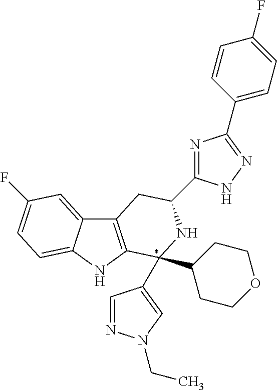 | 530 (m/e) | 1.03 | Diastereomer 2 at * carbon |
| 76 | 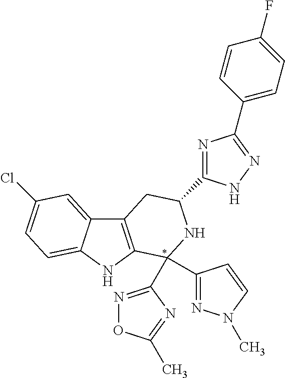 | 530 (m/e) | 1.15 | Diastereomer 1 at * carbon |
| 77 | 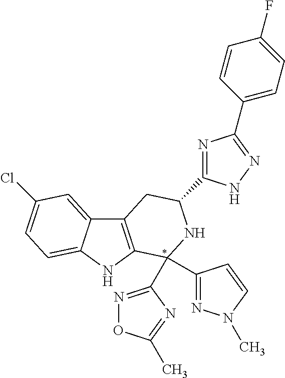 | 530 (m/e) | 1.14 | Diastereomer 2 at * carbon |
| 78 | 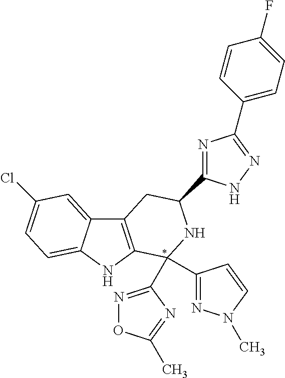 | 530 (m/e) | 1.14 | Diastereomer 1 at * carbon |
| 79 | 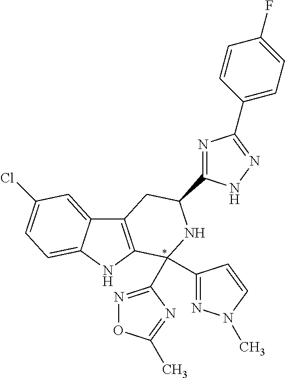 | 530 (m/e) | 1.14 | Diastereomer 2 at * carbon |

### Example of a Pharmaceutical Formulation

As a specific embodiment of an oral composition of a compound of the present invention, 50 mg of the compound of any of the Examples is formulated with sufficient finely divided lactose to provide a total amount of 580 to 590 mg to fill a size O hard gelatin capsule.

As a second specific embodiment of an oral composition of a compound of the present invention, 100 mg of the compound of any of the Examples, microcrystalline cellulose (124 mg), croscarmellose sodium (8 mg), and anhydrous unmilled dibasic calcium phosphate (124 mg) are thoroughly mixed in a blender; magnesium stearate (4 mg) and sodium stearyl fumarate (12 mg) are then added to the blender, mixed, and the mix transferred to a rotary tablet press for direct compression. The resulting tablets are optionally film-coated with Opadry® II for taste masking.

While the invention has been described and illustrated in reference to specific embodiments thereof, those skilled in the art will appreciate that various changes, modifications, and substitutions can be made therein without departing from the spirit and scope of the invention. For example, effective dosages other than the preferred doses as set forth hereinabove may be applicable as a consequence of variations in the responsiveness of the human being treated for a particular condition. Likewise, the pharmacologic response observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended therefore that the invention be limited only by the scope of the claims which follow and that such claims be interpreted as broadly as is reasonable.

1. A compound of structural formula I: 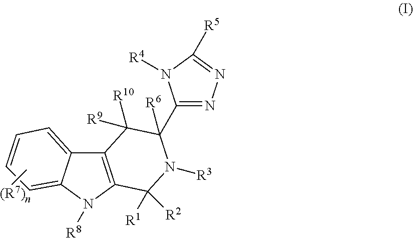
or a pharmaceutically acceptable salt thereof, wherein:
R¹ is selected from the group consisting of:
(1) C₁₋₁₀ alkyl,
(2) —C(O)OR^{e},
(3) —C(O)NR^{c}R^{d},
(4) C₂₋₁₀ cycloheteroalkyl,
(5) C₂₋₁₀ cycloheteroalkyl-C₁₋₁₀ alkyl-,
(6) aryl,
(7) heteroaryl, and
(8) heteroaryl-C₁₋₁₀ alkyl-,
wherein alkyl and cycloheteroalkyl are optionally substituted with one to three substituents independently selected from R^{a}, and aryl and heteroaryl are optionally substituted with one to three substituents independently selected from R^{b};
R² is selected from the group consisting of
(1) hydrogen,
(2) C₁₋₁₀ alkyl,
(3) C₂₋₁₀ alkenyl,
(4) C₂₋₁₀ alkynyl,
(5) C₃₋₁₀ cycloalkyl,
(6) C₃₋₁₀ cycloalkyl-C₁₋₁₀ alkyl-,
(7) C₁₋₆ alkyl-X—C₁₋₆ alkyl-,
(8) C₃₋₁₀ cycloalkyl-X—C₁₋₆ alkyl-,
(9) C₂₋₁₀ cycloheteroalkyl,
(10) aryl,
(11) hetero aryl,
(12) heteroaryl-C₁₋₆ alkyl-,
(13) aryl-C₁₋₄ alkyl-X—C₁₋₄ alkyl-, and
(14) heteroaryl-C₁₋₄ alkyl-X—C₁₋₄ alkyl-,
wherein X is selected from the group consisting of oxygen, sulfur, and NR⁴, and alkyl, alkenyl, alkynyl are optionally substituted with one to three substituents independently selected from R^{a}, and cycloalkyl, cycloheteroalkyl, aryl and heteroaryl are optionally substituted with one to three substituents independently selected from R^{b};
R³ is selected from the group consisting of
(1) hydrogen,
(2) —C₁₋₁₀ alkyl,
(3) —C₃₋₁₀ cycloalkyl,
(4) —C₂₋₁₀ cycloheteroalkyl,
(5) —C₂₋₁₀ cycloheteroalkyl-C₁₋₆ alkyl-, and
(6) heteroaryl-C₁₋₆ alkyl-,
wherein alkyl, cycloalkyl, and cycloheteroalkyl are optionally substituted with one to three substituents independently selected from R^{a}, and heteroaryl is optionally substituted with one to three substituents independently selected from R^{b};
R⁴ is selected from:
(1) hydrogen, and
(2) —C₁₋₁₀ alkyl, optionally substituted with one to five fluorines;
R⁵ is independently selected from the group consisting of
(1) hydrogen,
(2) —C₁₋₁₀ alkyl,
(3) —C₂₋₁₀ alkenyl,
(4) —C₂₋₁₀ alkynyl,
(5) —C₃₋₁₀ cycloalkyl,
(6) —C₂₋₁₀ cycloheteroalkyl,
(7) aryl, and
(8) heteroaryl,
wherein alkyl, alkenyl, alkynyl, cycloalkyl, and cycloheteroalkyl are optionally substituted with one to three substituents independently selected from R^{a}, and aryl and heteroaryl are optionally substituted with one to three substituents independently selected from R^{b};
R⁶ is selected from the group consisting of:
(1) hydrogen,
(2) —C₁₋₁₀ alkyl, optionally substituted with one to five fluorines,
(3) —C₂₋₁₀ alkenyl,
(4) —C₃₋₁₀ cycloalkyl, and
(5) —C₁₋₄ alkyl-O—C₁₋₄ alkyl-;
each R⁷ is independently selected from the group consisting of:
(1) hydrogen,
(2) —OR^{e},
(3) —NR^{c}S(O)ₘR^{e},
(4) halogen,
(5) —S(O)ₘR^{e},
(6) —S(O)ₘNR^{c}R^{d},
(7) —NR^{c}R^{d},
(8) —C(O)R^{e},
(9) —OC(O)R^{e},
(10) —CO₂R^{e},
(11) —CN,
(12) —C(O)NR^{c}R^{d},
(13) —NR^{c}C(O)R^{e},
(14) —NR^{c}C(O)OR^{e},
(15) —NR^{c}C(O)NR^{c}R^{d},
(16) —OCF₃,
(17) —OCHF₂,
(18) —C₂₋₁₀ cycloheteroalkyl,
(19) —C₁₋₁₀ alkyl, optionally substituted with one to five fluorines,
(20) —C₃₋₆ cycloalkyl,
(21) aryl, and
(22) heteroaryl,
wherein aryl and heteroaryl are optionally substituted with one to three substituents independently selected from R^{b};
R⁸ is selected from the group consisting of
(1) hydrogen,
(2) —C₁₋₁₀ alkyl,
(3) —C₂₋₁₀ alkenyl, and
(4) —C₃₋₁₀ cycloalkyl,
wherein alkyl, alkenyl, and cycloalkyl are optionally substituted with one to three substituents independently selected from R^{a};
R⁹ and R¹⁰ are each independently selected from:
(1) hydrogen, and
(2) —C₁₋₄ alkyl, optionally substituted with one to five fluorines;
each R^{a} is independently selected from the group consisting of:
(1) —OR^{e},
(2) —NR^{c}S(O)ₘR^{e},
(3) halogen,
(4) —S(O)ₘR^{e},
(5) —S(O)ₘNR^{c}R^{d},
(6) —NR^{c}R^{d},
(7) —C(O)R^{e},
(8) —OC(O)R^{e},
(9) oxo,
(10) —CO₂R^{e},
(11) —CN,
(12) —C(O)NR^{c}R^{d},
(13) —NR^{c}C(O)R^{e},
(14) —NR^{c}C(O)OR^{e},
(15) —NR^{c}C(O)NR^{c}R^{d},
(16) —CF₃,
(17) —OCF₃,
(18) —OCHF₂, and
(19) —C₂₋₁₀ cycloheteroalkyl;
each R^{b} is independently selected from the group consisting of:
(1) R^{a},
(2) C₁₋₁₀ alkyl, and
(3) C₃₋₆ cycloalkyl;
R^{c} and R^{d} are each independently selected from the group consisting of:
(1) hydrogen,
(2) —C₁₋₁₀ alkyl,
(3) —C₂₋₁₀ alkenyl,
(4) —C₃₋₆ cycloalkyl,
(5) —C₃₋₆ cycloalkyl-C₁₋₁₀ alkyl-,
(6) C₂₋₁₀ cycloheteroalkyl,
(7) C₂₋₁₀ cycloheteroalkyl-C₁₋₁₀ alkyl-,
(8) aryl,
(9) heteroaryl,
(10) aryl-C₁₋₁₀ alkyl-, and
(11) heteroaryl-C₁₋₁₀ alkyl-, or
R^{c} and R^{d} together with the atom(s) to which they are attached form a heterocyclic ring of 4 to 7 members containing 0-2 additional heteroatoms independently selected from oxygen, sulfur and N—R^{g} when R^{c} and R^{d} are other than hydrogen, and wherein each R^{c} and R^{d} is optionally substituted with one to three substituents independently selected from R^{h};
each R^{e} is independently selected from the group consisting of:
(1) hydrogen,
(2) —C₁₋₁₀ alkyl,
(3) —C₂₋₁₀ alkenyl,
(4) —C₃₋₆ cycloalkyl,
(5) —C₃₋₆ cycloalkyl-C₁₋₁₀ alkyl-,
(6) C₂₋₁₀ cycloheteroalkyl,
(7) C₂₋₁₀ cycloheteroalkyl-C₁₋₁₀ alkyl-,
(8) aryl,
(9) heteroaryl,
(10) aryl-C₁₋₁₀ alkyl-, and
(11) heteroaryl-C₁₋₁₀ alkyl-,
wherein when R^{e} is not hydrogen, each R^{e} is optionally substituted with one to three substituents selected from R^{h};
each R^{g} is independently selected from:
(1) —C(O)R^{e}, and
(2) —C₁₋₁₀ alkyl, optionally substituted with one to five fluorines;
each R^{h} is independently selected from the group consisting of:
(1) halogen,
(2) —C₁₋₁₀ alkyl,
(3) —O—C₁₋₄ alkyl,
(4) —S(O)ₘ—C₁₋₄ alkyl,
(5) —CN,
(6) —CF₃,
(7) —OCHF₂, and
(8) —OCF₃;
each m is independently 0, 1 or 2; and
each n is independently 0, 1, 2 or 3.

2. The compound of claim 1 wherein R³, R⁴, R⁶, R⁸, R⁹, and R¹⁰ are each hydrogen; or a pharmaceutically acceptable salt thereof.

3. The compound of claim 2 wherein R⁵ is aryl, wherein aryl is unsubstituted or substituted with one to three substituents independently selected from R^{b}; or a pharmaceutically acceptable salt thereof.

4. The compound of claim 2 wherein R⁵ is phenyl, wherein phenyl is unsubstituted or substituted with one to three substituents independently selected from halogen;
or a pharmaceutically acceptable salt thereof.

5. The compound of claim 2 wherein R⁵ is selected from the group consisting of:
(1) phenyl,
(2) para-fluorophenyl, and
(3) meta-fluorophenyl;
or a pharmaceutically acceptable salt thereof.

6. The compound of claim 2 wherein each R⁷ is independently selected from the group consisting of:
(1) hydrogen,
(2) halogen, and
(3) —CN;
or a pharmaceutically acceptable salt thereof.

7. The compound of claim 1 wherein n is 0 or 1; or a pharmaceutically acceptable salt thereof.

8. The compound of claim 1 wherein R¹ is selected from the group consisting of:
(1) C₁₋₁₀ alkyl,
(2) aryl, and
(3) heteroaryl,
wherein alkyl is unsubstituted or substituted with one to three substituents independently selected from R^{a}, and aryl and heteroaryl are unsubstituted or substituted with one to three substituents independently selected from R^{b}; or a pharmaceutically acceptable salt thereof.

9. The compound of claim 8 wherein R¹ is heteroaryl, wherein heteroaryl is unsubstituted or substituted with one to three substituents independently selected from R^{b}; or a pharmaceutically acceptable salt thereof.

10. The compound of claim 1 wherein R² is selected from the group consisting of:
(1) hydrogen,
(2) C₁₋₁₀ alkyl,
(3) C₃₋₁₀ cycloalkyl,
(4) C₂₋₁₀ cycloheteroalkyl,
(5) aryl, and
(6) heteroaryl,
wherein alkyl is unsubstituted or substituted with one to three substituents independently selected from R^{a}, and cycloalkyl, cycloheteroalkyl, aryl and heteroaryl are unsubstituted or substituted with one to three substituents independently selected from R^{b}; or a pharmaceutically acceptable salt thereof.

11. The compound of claim 10 wherein R² is selected from the group consisting of
(1) C₁₋₁₀ alkyl,
(2) C₂₋₆ cycloheteroalkyl,
(3) aryl, and
(4) heteroaryl,
wherein alkyl is unsubstituted or substituted with one to three substituents independently selected from R^{a}, and cycloheteroalkyl, aryl and heteroaryl are unsubstituted or substituted with one to three substituents independently selected from R^{b}; or a pharmaceutically acceptable salt thereof.

12. The compound of claim 1 wherein
R¹ is heteroaryl, wherein heteroaryl is unsubstituted or substituted with one to three substituents independently selected from R^{b};
R² is selected from the group consisting of
(1) C₁₋₁₀ alkyl,
(2) C₂₋₆ cycloheteroalkyl,
(3) aryl, and
(4) heteroaryl,
wherein alkyl is unsubstituted or substituted with one to three substituents independently selected from R^{a}, and cycloheteroalkyl, aryl and heteroaryl are unsubstituted or substituted with one to three substituents independently selected from R^{b};
R³, R⁴, R⁶, R⁸, R⁹, and R¹⁰ are hydrogen;
R⁵ is phenyl, wherein phenyl is unsubstituted or substituted with one to three substituents independently selected from halogen;
R⁷ is independently selected from the group consisting of:
(1) hydrogen,
(2) halogen, and
(3) —CN; and
n is 0 or 1;
or a pharmaceutically acceptable salt thereof.

13. The compound of claim 12 selected from the group consisting of: 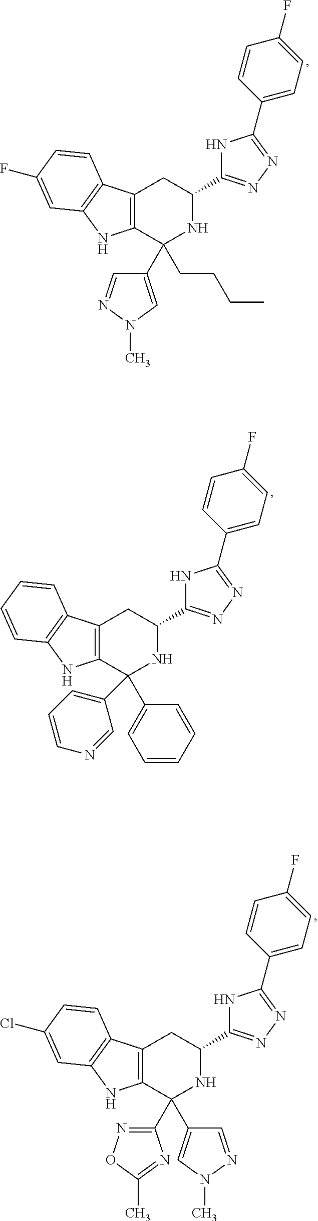 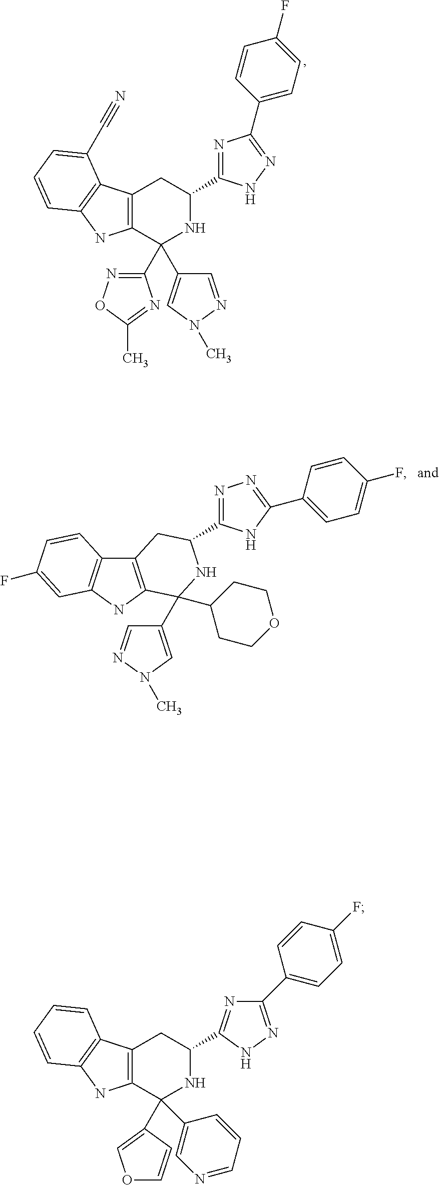
or a pharmaceutically acceptable salt thereof.

14. A pharmaceutical composition comprising a compound in accordance with claim 1 in combination with a pharmaceutically acceptable carrier.

15-17. (canceled)

18. A method of treating a disorder, condition, or disease responsive to antagonism of the somatostatin subtype receptor 3 in a mammal in need thereof comprising administration of a therapeutically effective amount of a compound according to claim 1.

19. The method of claim 18 wherein the disorder, condition, or disease is selected from the group consisting of: Type 2 diabetes, insulin resistance, hyperglycemia, obesity, a lipid disorder, Metabolic Syndrome, and hypertension.
